(19) United States
(12) Patent Application Publication
(10) Pub. No.: US 2009/0325264 A1
(43) Pub. Date: Dec. 31, 2009

(54) Engineered Delta-15-Fatty Acid Desaturases

(76) Inventors: MICHAEL J. STOREK, WALTHAM, MA (US); HENRY E. VALENTIN, DAVIS, CA (US); STEVEN E. SCREEN, ST. LOUIS, MO (US); VIRGINIA URSIN, PAWCATUCK, CT (US); BYRON FROMAN, DAVIS, CA (US); KEVIN JARRELL, LINCOLN, MA (US); SARA A. SALVADOR, SUDBURY, MA (US); ROBERT MCCARROLL, LEXINGTON, MA (US); PRASHANTH VISHWANATH, ARLINGTON, MA (US)

Correspondence Address: MONSANTO COMPANY, 800 N. LINDBERGH BLVD., ATTENTION: GAIL P. WUELLNER, IP PARALEGAL, (E1NA), ST. LOUIS, MO, 63167, US

(21) Appl. No.: 12/428,820

(22) Filed: Apr. 23, 2009

Related U.S. Application Data
(60) Provisional application No. 61/048,248, filed on Apr. 28, 2008.

(51) Int. Cl. C12N 9/02; C07H 21/00; C12N 5/04; C12N 5/10

(52) U.S. Cl. 435/189; 536/23.2; 435/419; 435/416; 435/412; 435/415; 435/414

(57) ABSTRACT

The present invention provides engineered fatty acid desaturase molecules preferring Gamma Linolenic Acid (GLA) over Linoleic Acid (LA) as a substrate. The invention further discloses compositions, polynucleotide constructs, transformed host cells, transgenic plants and seeds comprising the desaturase molecule, and methods for preparing and using the same. In particular, the disclosed engineered desaturase molecules are capable of altering the omega-3 fatty acid profiles in plants and plant parts.

This application claims benefit under 35USC§ 119(e) of U.S. provisional application Ser. No. 61/048,248 filed Apr. 28, 2008, herein incorporated by reference in its entirety.

### INCORPORATION OF SEQUENCE LISTING

A sequence listing containing the file named pa_{—}01220.txt, which is 2,296,089 bytes (as measured in Microsoft Windows®) and created on Apr. 23, 2009, comprises 902 polynucleotide and protein sequences, and is herein incorporated by reference in its entirety.

### FIELD OF THE INVENTION

The invention relates generally to desaturase enzymes that modulate the number and location of double bonds in long chain polyunsaturated fatty acids (LC-PUFAs), methods of use thereof, methods of generating such molecules, and compositions derived therefrom. In particular, the invention relates to engineered delta-15 desaturase enzymes that exhibit improved properties, and nucleic acids encoding for such enzymes.

### BACKGROUND

The primary products of fatty acid biosynthesis in most organisms are 16- and 18-carbon compounds. The relative ratio of chain lengths and degree of unsaturation of these fatty acids vary widely among species. Mammals, for example, produce primarily saturated and monosaturated fatty acids, while most higher plants produce fatty acids with one, two, or three double bonds, the latter two comprising polyunsaturated fatty acids (PUFAs).

Two main families of PUFAs are the omega-3 fatty acids (also represented as “n-3” fatty acids), exemplified by eicosapentaenoic acid (EPA, 20:4, n-3), and the omega-6 fatty acids (also represented as “n-6” fatty acids), exemplified by arachidonic acid (ARA, 20:4, n-6). PUFAs are important components of the plasma membrane of the cell and adipose tissue, where they may be found in such forms as phospholipids and as triglycerides, respectively. PUFAs are necessary for proper development in mammals, particularly in the developing infant brain, and for tissue formation and repair.

Several disorders respond to treatment with fatty acids. Supplementation with PUFAs has been shown to reduce the rate of restenosis after angioplasty (see, e.g., Bairati et al. 1992). The health benefits of certain dietary omega-3 fatty acids for cardiovascular disease and rheumatoid arthritis also have been well documented (see, e.g., Simopoulos, 1997; Cleland and James, 2000). Administration of stearidonic acid (SDA), an omega-3 fatty acid, has been shown to inhibit biosynthesis of leukotrienes (U.S. Pat. No. 5,158,975, herein incorporated by reference in its entirety). The consumption of SDA has been shown to lead to a decrease in blood levels of proinflammatory cytokines TNF-α and IL-1β (WO/03075670, herein incorporated by reference in its entirety).

Dietary consumption of long chain omega-3 fatty acids have been shown to impart health benefits. With this base of evidence, health authorities and nutritionists in Canada (Scientific Review Committee, 1990, Nutrition Recommendations, Minister of National Health and Welfare, Canada, Ottowa), Europe (de Deckerer et al., 1998), the United Kingdom (The British Nutrition Foundation, 1992, Unsaturated fatty-acids—nutritional and physiological significance: The report of the British Nutrition Foundation's Task Force, Chapman and Hall, London), and the United States (Simopoulos et al., 1999) have recommended increased dietary consumption of these PUFAs.

PUFAs, such as linoleic acid (LA, 18:2, Δ9, 12) and α-linolenic acid (ALA, 18:3, Δ9, 12, 15), are regarded as essential fatty acids in the diet because mammals lack the ability to synthesize these acids. LA is produced from oleic acid (OA, 18:1, Δ9) by a Δ12-desaturase while ALA is produced from LA by a Δ15-desaturase. When ingested, mammals have the ability to metabolize LA and ALA to form the n-6 and n-3 families of long LC-PUFAs. These LC-PUFAs are important cellular components conferring fluidity to membranes and functioning as precursors of biologically active eicosanoids such as prostaglandins, prostacyclins, and leukotrienes, which regulate normal physiological functions. ARA (20:4, n-6) is the principal precursor for the synthesis of eicosanoids, which include leukotrienes, prostaglandins, and thromboxanes, and which also play a role in the inflammation process.

However, mammals cannot synthesize essential PUFAs and can only obtain them in their diet. In mammals, the formation of certain LC-PUFAs is rate-limited by the step of Δ6 desaturation, which converts LA to GLA and ALA to SDA. Many physiological and pathological conditions have been shown to depress this metabolic step even further, and consequently, the production of LC-PUFAs. To overcome the rate-limiting step and increase tissue levels of EPA, one could consume large amounts of ALA. However, consumption of just moderate amounts of SDA provides an efficient source of EPA, as SDA is about four times more efficient than ALA at elevating tissue EPA levels in humans (U.S. Pat. No. 7,163,960, herein incorporated by reference in its entirety). In the same studies, SDA administration was also able to increase the tissue levels of docosapentaenoic acid (DPA), which is an elongation product of EPA. Alternatively, bypassing the Δ6-desaturation via dietary supplementation with EPA or Docosahexaenoic acid (DHA) can effectively alleviate many pathological diseases associated with low levels of PUFAs.

The need for a reliable and economical source of PUFAs has spurred interest in alternative sources of PUFAs. However, currently available sources of PUFAs are not desirable for a multitude of reasons. There are several disadvantages associated with commercial production of PUFAs from natural sources. Natural sources of PUFAs, such as animals and plants, have limited source supplies and tend to have highly heterogeneous oil compositions. The oils obtained from these sources can require extensive purification to separate out one or more desired PUFAs or to produce an oil that is enriched in one or more PUFAs.

Major long chain PUFAs of importance include DHA and EPA, which are primarily found in different types of fish oil, and ARA, found in filamentous fungi such as Mortierella. For DHA, a number of sources exist for commercial production including a variety of marine organisms, oils obtained from cold water marine fish, and egg yolk fractions. Commercial sources of SDA include the plant genera Trichodesma, Borago (borage) and Echium. Natural sources of PUFAs also are subject to uncontrollable fluctuations in availability. Fish stocks may undergo natural variation or may be depleted by overfishing. In addition, even with overwhelming evidence of their therapeutic benefits, dietary recommendations regarding omega-3 fatty acids are not heeded. Fish oils have unpleasant tastes and odors, which may be impossible to economically separate from the desired product, and can render such products unacceptable as food supplements. Animal oils, and particularly fish oils, can accumulate environmental pollutants. Foods may be enriched with fish oils, but again, such enrichment is problematic because of cost and declining fish stocks worldwide. This problem is also an impediment to consumption and intake of whole fish. Nonetheless, if the health messages to increase fish intake were embraced by communities, there would likely be a problem in meeting demand for fish. Furthermore, there are problems with sustainability of this industry, which relies heavily on wild fish stocks for aquaculture feed (Naylor et al., 2000). Large scale fermentation of organisms is expensive. Natural animal tissues contain low amounts of ARA and are difficult to process. Furthermore, the use of desaturase molecules derived from Caenorhabditis elegans (Meesapyodsuk et al., 2000) is not ideal for the commercial production of enriched plant seed oils.

Therefore, it would be advantageous to obtain or design genetic material involved in PUFA biosynthesis and to express the isolated material in a plant system, in particular, a land-based terrestrial crop plant system, that can be manipulated to provide production of commercial quantities of one or more PUFAs. There is also a need to increase omega-3 fat intake in humans and animals. Thus there is a need to provide a wide range of omega-3 enriched foods and food supplements so that subjects can choose feed, feed ingredients, food and food ingredients that suit their usual dietary habits. Currently there is only one omega-3 fatty acid, ALA, available in vegetable oils. However, there is poor conversion of ingested ALA to the longer-chain omega-3 fatty acids such as EPA and DHA. It has been demonstrated in U.S. Pat. No. 7,163,960 (herein incorporated by reference in its entirety) for “Treatment And Prevention Of Inflammatory Disorders,” that elevating ALA intake from the community average of 1/g day to 14 g/day by use of flaxseed oil only modestly increased plasma phospholipid EPA levels. A 14-fold increase in ALA intake resulted in a 2-fold increase in plasma phospholipid EPA (Mantzioris et al., 1994).

Based on studies, it is seen that in commercial oilseed crops, such as canola, soybean, corn, sunflower, safflower, or flax, the conversion of some fraction of the mono- and polyunsaturated fatty acids that typify their seed oil to SDA requires the seed-specific expression of multiple desaturase enzymes, including Δ6- and Δ12, and an enzyme that has Δ15-desaturase activity. Oils derived from plants expressing elevated levels of Δ6, Δ12, and Δ15-desaturases are rich in SDA and other omega-3 fatty acids. Such oils can be utilized to produce foods and food supplements enriched in omega-3 fatty acids and consumption of such foods effectively increases tissue levels of EPA and DHA. Foods and food stuffs, such as milk, margarine and sausages, made or prepared with omega-3 enriched oils will result in therapeutic benefits. Thus, novel nucleic acids of Δ15-desaturases for use in transgenic crop plants would be desirable, to produce oils enriched in PUFAs. New plant seed oils enriched for PUFAs and, particular, omega-3 fatty acids such as stearodonic acid, would be similarly useful.

To that end, an efficient and commercially viable production of PUFAs using fatty acid desaturases, genes encoding them, and recombinant methods of producing them, would be highly desirable. Additionally useful would be oils containing higher relative proportions of and/or enriched in specific PUFAs and food compositions and supplements containing them, as well as for reliable economical methods of producing specific PUFAs.

### SUMMARY OF THE INVENTION

In one aspect, the invention provides engineered molecules that desaturate a fatty acid molecule at carbon 15 (Δ15-desaturase), and polynucleotides encoding such molecules. These may be used to transform cells or modify the fatty acid composition of a plant or the oil produced by a plant. One embodiment of the invention is an engineered Δ15-desaturase molecule that exhibits a high conversion rate of GLA to SDA and a substrate preference for GLA over LA. Another embodiment is a polynucleotide molecule encoding such a desaturase molecule. Yet another embodiment is a construct, plant cell, transgenic plant, progeny of said plant or seed of said plant comprising said engineered desaturase molecule. A further embodiment is a method of producing or using said engineered desaturase molecule.

The present invention provides a desaturase molecule that exhibits a substrate preference for GLA over LA, as evidenced by the SDA/ALA ratio, of at least 1.6×, at least 1.65×, at least 1.7×, at least 1.75×, 1.8×, 1.9×, 2.0× or even greater such as at least 2.5×, at least 5.0× or at least 7.5×.

In other embodiments, the present invention provides a desaturase molecule that exhibits a total conversion rate of GLA to SDA of at least 40%, at least 41%, at least 42%, at least 43%, at least 44%, at least 45%, at least 46%, at least 47%, or even greater such as at least 50%, at least 55% or at least 60%.

Another aspect of the present invention is a desaturase molecule, that when expressed in a transgenic plant, causes the transgenic plant to produce more omega-3 fatty acid compared to that of a non-transgenic plant. Another aspect of the present invention is a desaturase molecule, that when expressed in a transgenic plant, causes the transgenic plant to produce more delta-6 desaturated omega-3 fatty acid compared to that of a non-transgenic plant.

Additional aspects of the present invention include methods for generating engineered desaturase molecule polypeptides and polynucleotides disclosed herein. Such engineered molecules are generated from the identification and manipulation of phenotypically important regions identified from a parental desaturase molecules. Such regions may include, but are not limited to, primary sequence motifs and secondary structures such as alpha helices or beta strands. Included in the present invention are alterations in molecular structure in polypeptide motifs of a parental fungal desaturase.

In another aspect, the invention provides an isolated polypeptide comprising a sequence selected from the group consisting of SEQ ID NO: 1 through 331, and polynucleotides encoding the same. In another aspect, the invention provides an isolated polynucleotide comprising a sequence selected from the group consisting of SEQ ID NO: 332 through 662. Further aspects of the present invention include engineered desaturase molecules that are derived from a parental molecule, or a molecule exhibiting 75%, 80%, 85%, 90%, 95% or 99% similarity to a fungal desaturase.

In yet another aspect, the invention provides a recombinant vector comprising an isolated polynucleotide in accordance with the invention. In still yet another aspect, the invention provides cells, such as mammalian, plant, insect, yeast and bacterial cells transformed with the polynucleotides of the instant invention. In a further embodiment, the cells are transformed with recombinant vectors comprising constitutive or tissue-specific promoters in addition to the polynucleotides of the present invention. In certain embodiments of the invention, such cells may also be defined as transformed with a nucleic acid sequence encoding a polypeptide having desaturase activity that desaturates a fatty acid molecule at carbon 6.

Still yet another aspect of the invention provides a method of producing seed oil comprising omega-3 fatty acids from plant seeds, comprising the steps of (a) obtaining seeds of a plant according to the invention; and (b) extracting the oil from said seeds. Examples of such a plant seed include canola, soy, soybeans, rapeseed, sunflower, cotton, cocoa, peanut, safflower, coconut, flax, oil palm, oilseed Brassica napus, and corn. Preferred methods of transforming such plant cells include the use of Ti and Ri plasmids of Agrobacterium, electroporation, and high-velocity ballistic bombardment.

In an additional aspect, a method is provided of producing a plant comprising seed oil containing altered levels of omega-3 fatty acids comprising introducing a recombinant vector of the invention into an oil-producing plant. In the method, introducing the recombinant vector may comprise plant breeding and may comprise the steps of: (a) transforming a plant cell with the recombinant vector; and (b) regenerating said plant from the plant cell, wherein the plant has altered levels of omega-3 fatty acids. In the method, the plant may, for example, be selected from the group consisting of Arabidopsis thaliana, oilseed Brassica, rapeseed, sunflower, safflower, canola, corn, soybean, cotton, flax, jojoba, Chinese tallow tree, tobacco, cocoa, peanut, fruit plants, citrus plants, and plants producing nuts and berries. The plant may be also defined as transformed with a nucleic acid sequence encoding a polypeptide having desaturase activity that desaturates a fatty acid molecule at carbon 6 and the plant may have SDA increased. The method may also further comprise introducing the recombinant vector into a plurality of oil-producing plants and screening the plants or progeny thereof having inherited the recombinant vector for a plant having a desired profile of omega-3 fatty acids.

In yet another aspect, the invention provides an endogenous seed oil having a SDA content of from about 8% to about 50% and an oleic acid content of from about 40% to about 75%. In certain embodiments, the seed oil may be further defined as comprising less than 10% combined ALA, LA and GLA. The oil may also comprise a SDA content further defined as from about 10% to about 35%, including from about 12% to about 35%, and about 15% to about 35%. In further embodiments of the invention, the seed oil may have an oleic acid content further defined as from about 45% to about 65%, including from about 50% to about 65%, from about 50% to about 60% and from about 55% to about 65%. In still further embodiments of the invention, the SDA content is further defined as from about 12% to about 35% and the oleic acid content is further defined as from about 55% to about 65%.

In still yet another aspect, the invention provides a method of increasing the nutritional value of an edible product for human or animal consumption, comprising adding a seed oil provided by the invention to the edible product. In certain embodiments, the product is human and/or animal food. The edible product may also be animal feed and/or a food supplement. In the method, the seed oil may increase the SDA content of the edible product and/or may decrease the ratio of omega-6 to omega-3 fatty acids of the edible product. The edible product may lack SDA prior to adding the seed oil.

In still yet another aspect, the invention provides a method of manufacturing food or feed, comprising adding a seed oil provided by the present invention to starting food or feed ingredients to produce the food or feed. The invention also provides food or feed made by the method.

In still yet another aspect, the invention comprises a method of providing SDA to a human or animal, comprising administering the seed oil provided by the present invention to said human or animal. In the method, the seed oil may be administered in an edible composition, including food or feed. Examples of food include, but are not limited to, beverages, infused foods, sauces, condiments, salad dressings, fruit juices, syrups, desserts, icings and fillings, soft frozen products, confections or intermediate food. The edible composition may be substantially a liquid or solid. The edible composition may also be a food supplement and/or nutraceutical. In the method, the seed oil may be administered to a human and/or an animal. Examples of animals the oil may be administered to include livestock or poultry.

Certain aspects of the present invention are described in the following statements:
Statement 1: An engineered fatty acid desaturase molecule, wherein said desaturase molecule:
   a. exhibits a substrate preference for Gamma Linolenic Acid (GLA) over Linoleic Acid (LA) of at least 1.75× and as calculated by the formula (SDA/(SDA+GLA))/(ALA/(LA+ALA), where SDA is stearodonic acid, GLA is gamma linolenic acid, ALA is alpha linolenic acid, and LA is linoleic acid; or
   b. exhibits a total conversion rate of GLA to SDA of at least 40%; or
   c. when expressed in a transgenic plant, causes the transgenic plant to produce more omega-3 fatty acid than non-transgenic plants; or
   d. when co-expressed with a delta-6 fatty acid desaturase in a transgenic plant, causes the transgenic plant to accumulate, as compared to a non-transgenic plant, a condition selected from the group consisting of: more SDA than ALA, and greater conversion of GLA to SDA than LA to ALA.
Statement 2: The desaturase molecule of statement 1, further defined as a molecule that desaturates a fatty acid molecule at carbon 15.
Statement 3: The desaturase molecule of statement 1, wherein said molecule has 80% similarity to a fungal desaturase.
Statement 4: The desaturase molecule of statement 1, wherein said molecule comprises amino acid sequence variants generated from a parental fungal desaturase.
Statement 5: The desaturase molecule of statement 1, wherein said desaturase is identified from a genus selected from the group consisting of: Mortierella, Neurospora, Aspergillus, Saccharomyces, Botrytis, Chlorella.
Statement 6: The desaturase molecule of statement 1, wherein the molecule has a sequence selected from the group consisting of SEQ ID NO: 1 through SEQ ID NO: 331.
Statement 7: The desaturase molecule of statement 1, wherein the molecule exhibits a percent sequence identity of greater than about 90% identity with a molecule selected from the group consisting of: SEQ ID NO: 1 through SEQ ID NO: 331.
Statement 8: The desaturase molecule of statement 1, wherein the molecule comprises a fragment of SEQ ID NO: 1 through SEQ ID NO: 331.
Statement 9: A polynucleotide encoding the desaturase molecule of statement 1.
Statement 10: The polynucleotide of statement 9, wherein the polynucleotide has a sequence selected from the group consisting of SEQ ID NO: 332 through SEQ ID NO: 662.
Statement 11: The polynucleotide of statement 9 that, when under the control of a regulatory element, is capable of expression in a plant.
Statement 12: The polynucleotide of statement 9, or any complement thereof, or any fragment thereof, comprising a nucleic acid sequence that exhibits a substantial percent sequence identity of greater than about 90% to a sequence selected from the group consisting of SEQ ID NO: 332 through SEQ ID NO: 662.
Statement 13: A polynucleotide that hybridizes under stringent conditions with the polynucleotide of statement 9, or a complement thereof, or a fragment thereof.
Statement 14: A construct comprising the polynucleotide of statement 9.
Statement 15: The construct of statement 14, further comprising a second polynucleotide that is transcribable.
Statement 16: The construct of statement 15, wherein the second transcribable polynucleotide molecule is selected from the group consisting of: a non-coding regulatory element, a selectable marker, a gene encoding a second desaturase, and a gene of agronomic interest.
Statement 17: The construct of statement 16, wherein the gene of agronomic interest is a gene controlling the phenotype of a trait selected from the group consisting of: herbicide tolerance, insect control, modified yield, fungal disease resistance, virus resistance, nematode resistance, bacterial disease resistance, plant growth and development, starch production, modified oils production, high oil production, modified fatty acid content, high protein production, fruit ripening, enhanced animal and human nutrition, biopolymers, environmental stress resistance, pharmaceutical peptides and secretable peptides, improved processing traits, improved digestibility, enzyme production, flavor, nitrogen fixation, hybrid seed production, fiber production, and biofuel production.
Statement 18: A host cell stably transformed with the construct of statement 14.
Statement 19: The host cell of statement 17, further defined as a plant cell.
Statement 20: The host cell of statement 17, further defined as a fungal cell.
Statement 21: The host cell of statement 17, further defined as a bacterial cell.
Statement 22: A progeny of the host cell of statement 17, wherein said progeny has inherited the polynucleotide of said polynucleotide construct.
Statement 23: The plant cell of statement 19, wherein said plant cell is a cell of a plant selected from the group consisting of: Arabidopsis thaliana, Brassica napus, Brassica rapa, rapeseed, sunflower, safflower, canola, corn, soybean, cotton, flax, jojoba, Chinese tallow tree, tobacco, cocoa, peanut, fruit plants, citrus plants, plants producing nuts, plants producing seeds, and plants producing berries.
Statement 24: A plant stably transformed with the polynucleotide of statement 9.
Statement 25: The plant of statement 24, wherein said plant is selected from the group consisting of: Arabidopsis thaliana, Brassica, rapeseed, sunflower, safflower, canola, corn, soybean, cotton, flax, jojoba, Chinese tallow tree, tobacco, cocoa, peanut, fruit plants, citrus plants, plants producing nuts, plants producing seeds, and plants producing berries.
Statement 26: A progeny of the plant of statement 24, wherein said progeny has inherited the polynucleotide of said polynucleotide construct.
Statement 27: A seed of said transgenic plant of statement 24.
Statement 28: A seed of said transgenic plant of statement 26.
Statement 29: A method of producing improved levels of stearodonic acid in a plant, comprising growing a transgenic plant comprising the desaturase molecule of statement 1, whereby the omega-3 fatty acid content of the seed is increased as compared to a seed of an isogenic plant lacking said desaturase molecule of statement 1.
Statement 30: A plant produced by the method of statement 29.
Statement 31: A progeny of the plant produced by the method of statement 29, wherein said progeny also exhibits the phenotype of increased omega-3 fatty acid production in the seed.
Statement 32: A seed of the plant of statement 30.
Statement 33: A method of producing improved levels of stearodonic acid in a plant, comprising growing a transgenic plant comprising a desaturase molecule selected from the group consisting of SEQ ID NO: 1 through SEQ ID NO: 331 whereby the omega-3 fatty acid content of the seed is increased as compared to a seed of an isogenic plant lacking said desaturase molecule.
Statement 34: A plant produced by the method of statement 33.
Statement 35: A progeny of the plant produced by the method of statement 32, wherein said progeny also exhibits the phenotype of increased omega-3 fatty acid production in the seed.
Statement 36: A seed of said plant of statement 34.
Statement 37: A method for selecting a delta-15 desaturase molecule producing improved levels of omega-3 fatty acids in yeast, comprising
   e. transforming a host cell with a transcribable polynucleotide encoding a desaturase molecule of statement 1;
   f. providing an appropriate substrate for said desaturase molecule to the yeast medium; and
   g. assaying the yeast culture for stearodonic acid production.
Statement 38: A method for assessing the oil composition of a seed of a plant comprising the desaturase of statement 1, comprising growing said plant, recovering a seed of said plant, extracting the oil molecules from said seed and assaying the oil composition.
Statement 39: A method for assessing the presence of the desaturase molecule of statement 1 in a plant or seed, comprising extracting said desaturase from a plant tissue.
Statement 40: A method for assaying stearodonic acid levels in plants, comprising extracting the stearodonic acid from a plant tissue.
Statement 41: A method of producing improved levels of stearodonic acid in a plant, comprising growing a transgenic plant comprising the desaturase molecule of statement 1, whereby the stearodonic acid content of the seed is increased as compared to a seed of an isogenic plant lacking said desaturase molecule of statement 1.
Statement 42: A method of producing food or feed, comprising the steps of:
   h. obtaining the plant of statement 24 or a part thereof; and
   i. producing said food or feed therefrom.
Statement 43: A food or feed composition produced by the method of statement 42.
Statement 44: A method of generating an enhanced desaturase, comprising:
   j. engineering a variant of a naturally-occurring desaturase; and
   k. analyzing the variants to identify those that:
      i. exhibits a substrate preference for Gamma Linolenic Acid (GLA) over Linoleic Acid (LA) of at least 1.75×, as measured in a yeast assay and as calculated by the formula (SDA/(SDA+GLA))/(ALA/(LA+ALA), where SDA is stearodonic acid, GLA is gamma linolenic acid, ALA is alpha linolenic acid, and LA is linoleic acid; or
      ii. exhibits a total conversion rate of GLA to SDA of at least 40%; or
      iii. when expressed in a transgenic plant, causes the transgenic plant to produce more omega-3 fatty acid than non-transgenic plants; or
      iv. when co-expressed with a delta-6 fatty acid desaturase in a transgenic plant, causes the transgenic plant to accumulate more SDA than ALA.
Statement 45: An isolated or recombinant polypeptide comprising an amino acid sequence with 90% sequence identity to the desaturase molecule of statement 1, wherein the amino acid sequence comprises at least one amino acid substitution or insertion in the putative alpha-helical region corresponding to positions 110-130, wherein the putative alpha-helical region is determined by MolSoft, ICMPRo or any comparable molecular modeling software.
Statement 46: An engineered polypeptide that exhibits delta-15 desaturase activity, wherein said polypeptide comprises a motif selected from the group consisting of:
   a. X₁X₂X₃X₄X₅NX₆X₇X₈, wherein Xᵢ represents a variable amino acid, wherein:
      (i) X₁ is selected from the group consisting of: D, R, E, P, N, Q, K and H; and
      (ii) X₂ is selected from the group consisting of: S, H, Y, N and P; and
      (iii) X₃ is selected from the group consisting of: K, N, Q, R and T; and
      (iv) X₄ is selected from the group consisting of: T, A, R, W and S; and
      (v) X₅ is selected from the group consisting of: I, F, V, W and L; and
      (vi) X₆ is selected from the group consisting of: T, D, N, Y and S; and
      (vii) X₇ is selected from the group consisting of: I, V, T and F; and
      (viii) X₈ is selected from the group consisting of: F, M, I and L;
   and
   b. X₉X₁₀X₁₁X₁₂X₁₃X₁₄X₁₅X₁₆X₁₇X₁₈X₁₉X₂₀, wherein Xᵢ represents a variable amino acid, wherein
      (i) X₉ is selected from the group consisting of: K, R and A; and
      (ii) X₁₀ is selected from the group consisting of: G, F, A, Y, N, D, V, C and S; and
      (iii) X₁₁ is selected from the group consisting of: T and H; and
      (iv) X₁₂ is selected from the group consisting of: G and N; and
      (v) X₁₃ is selected from the group consisting of: S, N, T, G, D, A, H, R and P; and
      (vi) X₁₄ is selected from the group consisting of: M, T and V; and
      (vii) X₁₅ is selected from the group consisting of: T, K, S, A and E; and
      (viii) X₁₆ is selected from the group consisting of: K, R and N; and
      (ix) X₁₇ is selected from the group consisting of: V, M, T, E, F, I and L; and
      (x) X₁₈ is selected from the group consisting of: V, A and S; and
      (xi) X₁₉ is selected from the group consisting of: F and W; and
      (xii) X₂₀ is selected from the group consisting of: I, V and H;
   and
   c. X₂₁X₂₂X₂₃X₂₄X₂₅SX₂₆X₂₇X₂₈X₂₉, wherein Xᵢ represents a variable amino acid, wherein
      (i) X₂₁ is selected from the group consisting of: P, R, K and S; and
      (ii) X₂₂ is selected from the group consisting of: D, R, E, G, S, N and K; and
      (iii) X₂₃ is selected from the group consisting of: V, L, T, Y, I and S; and
      (iv) X₂₄ is selected from the group consisting of: W, L, T, K, F, G, V, I, S and M; and
      (v) X₂₅ is selected from the group consisting of: I, K, L, W and R; and
      (vi) X₂₆ is selected from the group consisting of: M, S, I, F, L, A and T; and
      (vii) X₂₇ is selected from the group consisting of: A, L, W, H, Y, R, I, V, F and M; and
      (viii) X₂₈ is selected from the group consisting of: Y and H; and
      (ix) X₂₉ is selected from the group consisting of: F, V, L and T;
   and
   d. X₃₀X₃₁X₃₂X₃₃X₃₄X₃₅X₃₆X₃₇X₃₈X₃₉X₄₀, wherein Xᵢ represents a variable amino acid, wherein
      (i) X₃₀ is selected from the group consisting of: F, L, V, I and F; and
      (ii) X₃₁ is selected from the group consisting of: A, L, V, F, G and I; and
      (iii) X₃₂ is selected from the group consisting of: M, Y, T, V, A, N and S; and
      (iv) X₃₃ is selected from the group consisting of: A, I, V, L, T and S; and
      (v) X₃₄ is selected from the group consisting of: F, S, A, T and L; and
      (vi) X₃₅ is selected from the group consisting of: G, V, I, A and L; and
      (vii) X₃₆ is selected from the group consisting of: L, V, T and S; and
      (viii) X₃₇ is selected from the group consisting of: G, F, V, A, Y, L, C and W; and
      (ix) X₃₈ is selected from the group consisting of: Y, A, I, F and V; and
      (x) X₃₉ is selected from the group consisting of: L, F, C, V, G, A and W; and
      (xi) X₄₀ is selected from the group consisting of: A, G and L;
   and
   e. X₄₁X₄₂X₄₃X₄₄X₄₅X₄₆X₄₇X₄₈GX₄₉X₅₀, wherein Xᵢ represents a variable amino acid, wherein
      (i) X₄₁ is selected from the group consisting of: W, Y and C; and
      (ii) X₄₂ is selected from the group consisting of: A, T, I, P, N, S and L; and
      (iii) X₄₃ is selected from the group consisting of: L, A, T, I and S; and
      (iv) X₄₄ is selected from the group consisting of: Y, Q and F; and
      (v) X₄₅ is selected from the group consisting of: G, W, S and I; and
      (vi) X₄₆ is selected from the group consisting of: Y, F, I, V and L; and
      (vii) X₄₇ is selected from the group consisting of: L, M, I, V and F; and
      (viii) X₄₈ is selected from the group consisting of: Q, I and M; and
      (ix) X₄₉ is selected from the group consisting of: L, C, T, V, I, R, S, M, W and F; and
      (x) X₅₀ is selected from the group consisting of: V, T, F, M and I;
   and
   f. X₅₁X₅₂X₅₃X₅₄X₅₅X₅₆X₅₇X₅₈X₅₉X₆₀, wherein Xᵢ represents a variable amino acid, wherein
      (i) X₅₁ is selected from the group consisting of: T, P, V, R and Q; and
      (ii) X₅₂ is selected from the group consisting of: E, R, K, S, D, G and N; and
      (iii) X₅₃ is selected from the group consisting of: A, K, S, D, V, T, G, R and W; and
      (iv) X₅₄ is selected from the group consisting of: D, E, Y, F, V, H and L; and
      (v) X₅₅ is selected from the group consisting of: K, R, E, G, Y and F; and
      (vi) X₅₆ is selected from the group consisting of: N, D, G, A, I, S, P, H and T; and
      (vii) X₅₇ is selected from the group consisting of: L, E, Q, V, T, A, Y and W; and
      (viii) X₅₈ is selected from the group consisting of: R, P, L, M and E; and
      (ix) X₅₉ is selected from the group consisting of: K, P, A, L, T, N, H and D; and
      (x) X₆₀ is selected from the group consisting of: L, R, V, K and G;
   and
   g. X₆₁X₆₂X₆₃X₆₄X₆₅X₆₆X₆₇X₆₈X₆₉X₇₀, wherein Xᵢ represents a variable amino acid, wherein
      (i) X₆₁ is selected from the group consisting of: K, P, A, L, T, N, H and D; and
      (ii) X₆₂ is selected from the group consisting of: L, R, V, K and G; and
      (iii) X₆₃ is selected from the group consisting of: Y, E, D, F, H, N, T, S and A; and
      (iv) X₆₄ is selected from the group consisting of: M, F, K, V, L, H, N, D, Q, E, Y and I; and
      (v) X₆₅ is selected from the group consisting of: D, P, S, E, L, A and V; and
      (vi) X₆₆ is selected from the group consisting of: K, A, S, Y and D; and
      (vii) X₆₇ is selected from the group consisting of: V, E, A, R, L, M, F, I, W and G; and
      (viii) X₆₈ is selected from the group consisting of: E, T, W, L, D, V, F, Y, N, H, K and Q; and
      (ix) X₆₉ is selected from the group consisting of: E, A, F, K, S, N and D; and
      (x) X₇₀ is selected from the group consisting of: E and W;
   and
   h. X₇₁X₇₂X₇₃X₇₄X₇₅X₇₆X₇₇X₇₈X₇₉X₈₀X₈₁, wherein Xᵢ represents a variable amino acid, wherein
      (i) X₇₁ is selected from the group consisting of: Y, G, A and W; and
      (ii) X₇₂ is selected from the group consisting of: W, T, F, L, Y, N, I, S, K, Q, P and H; and
      (iii) X₇₃ is selected from the group consisting of: L, Q, P and F; and
      (iv) X₇₄ is selected from the group consisting of: M, G, L, F, V, I, S, A and T; and
      (v) X₇₅ is selected from the group consisting of: Y, A, S, T, G, W and R; and
      (vi) X₇₆ is selected from the group consisting of: L, I, V, F and T; and
      (vii) X₇₇ is selected from the group consisting of: L, C, T, A, K, I, V, F and T; and
      (viii) X₇₈ is selected from the group consisting of: F, A, T, N, I, S, L, M and V; and
      (ix) X₇₉ is selected from the group consisting of: N, Y, V, R, G, D, H, L and F; and
      (x) X₈₀ is selected from the group consisting of: V, L, I, A, W, Y, F, Q and E; and
      (xi) X₈₁ is selected from the group consisting of: S, T, P A and C;
   and
   i. X₈₂X₈₃X₈₄X₈₅X₈₆X₈₇X₈₈X₈₉X₉₀X₉₁X₉₂, wherein Xᵢ represents a variable amino acid, wherein
      (i) X₈₂ is selected from the group consisting of: V, G and S; and
      (ii) X₈₃ is selected from the group consisting of: K, N, D, Y, I, F and V; and
      (iii) X₈₄ is selected from the group consisting of: F, Q, I, L and V; and
      (iv) X₈₅ is selected from the group consisting of: S, G and T; and
      (v) X₈₆ is selected from the group consisting of: G, N, K, A, S and C; and
      (vi) X₈₇ is selected from the group consisting of: H, M, W, I, F, D, N, Y, G and R; and
      (vii) X₈₈ is selected from the group consisting of: E, G, K, T, A, N, D, R and S; and
      (viii) X₈₉ is selected from the group consisting of: A, G, S, C, E, R, T and K; and
      (ix) X₉₀ is selected from the group consisting of: P, W, Q, S, T and A; and
      (x) X₉₁ is selected from the group consisting of: H, L, Q, N and K; and
      (xi) X₉₂ is selected from the group consisting of: W, F, G, S and R;
   and
   j. X₉₃X₉₄X₉₅X₉₆X₉₇X₉₈X₉₉X₁₀₀X₁₀₁X₁₀₂X₁₀₃, wherein Xᵢ represents a variable amino acid, wherein
      (i) X₉₃ is selected from the group consisting of: F and Y; and
      (ii) X₉₄ is selected from the group consisting of: Q, E, D, S and W; and
      (iii) X₉₅ is selected from the group consisting of: T, P, S and A; and
      (iv) X₉₆ is selected from the group consisting of: V, I, G, S, A, T, K and Q; and
      (v) X₉₇ is selected from the group consisting of: P, A, S, T and D; and
      (vi) X₉₈ is selected from the group consisting of: L, V, I and F; and
      (vii) X₉₉ is selected from the group consisting of: Y, F, W and L; and
      (viii) X₁₀₀ is selected from the group consisting of: E, A, G, T, R, K, D and L; and
      (ix) X₁₀₁ is selected from the group consisting of: P, A, T, S, Q, H, K, N, D, E and R; and
      (x) X₁₀₂ is selected from the group consisting of: H, Q, N, K, S, R and E; and
      (xi) X₁₀₃ is selected from the group consisting of: Q, E and D;
   and
   k. X₁₀₄X₁₀₅X₁₀₆X₁₀₇X₁₀₈X₁₀₉X₁₁₀X₁₁₁X₁₁₂X₁₁₃X₁₁₄, wherein Xᵢ represents a variable amino acid, wherein
      (i) X₁₀₄ is selected from the group consisting of: R, A, S, F and G; and
      (ii) X₁₀₅ is selected from the group consisting of: K, H, I, W, P, S, V, N, M and R; and
      (iii) X₁₀₆ is selected from the group consisting of: N, L, D, W, Y, A and Q; and
      (iv) X₁₀₇ is selected from the group consisting of: I, V and C; and
      (v) X₁₀₈ is selected from the group consisting of: F, V, L, A, E and I; and
      (vi) X₁₀₉ is selected from the group consisting of: Y, I, L, M, T, V, W and A; and
      (vii) X₁₁₀ is selected from the group consisting of: S, L, V, F and W; and
      (viii) X₁₁₁ is selected from the group consisting of: N, D, L and G; and
      (ix) X₁₁₂ is selected from the group consisting of: C, I, L, K and G; and
      (x) X₁₁₃ is selected from the group consisting of: G, I, L, V, W, F and C; and
      (xi) X₁₁₄ is selected from the group consisting of: I, L, Q, W and C;
   and
   l. X₁₁₅X₁₁₆X₁₁₇X₁₁₈X₁₁₉X₁₂₀X₁₂₁X₁₂₂X₁₂₃, wherein Xᵢ represents a variable amino acid, wherein
      (i) X₁₁₅ is selected from the group consisting of: A, L, S and V; and
      (ii) X₁₁₆ is selected from the group consisting of: M, V, T, W, F and C; and
      (iii) X₁₁₇ is selected from the group consisting of: G, A, V, L, I and F; and
      (iv) X₁₁₈ is selected from the group consisting of: S, A, Y, F, L and G; and
      (v) X₁₁₉ is selected from the group consisting of: I, A, G and F; and
      (vi) X₁₂₀ is selected from the group consisting of: L, N, Y, A, I, F, V and H; and
      (vii) X₁₂₁ is selected from the group consisting of: T, W, Y, A, L, F and S; and
      (viii) X₁₂₂ is selected from the group consisting of: Y, Q, L, G, T, W and F; and
      (ix) X₁₂₃ is selected from the group consisting of: L, A, W, C and I;
   and
   m. X₁₂₄X₁₂₅X₁₂₆X₁₂₇X₁₂₈X₁₂₉, wherein Xᵢ represents a variable amino acid, wherein
      (i) X₁₂₄ is selected from the group consisting of: H, A, V, S, L, G and P; and
      (ii) X₁₂₅ is selected from the group consisting of: W, F and M; and
      (iii) X₁₂₆ is selected from the group consisting of: I, L, F and V; and
      (iv) X₁₂₇ is selected from the group consisting of: V, I, L, F, M and D; and
      (v) X₁₂₈ is selected from the group consisting of: C, A, F, V and I; and
      (vi) X₁₂₉ is selected from the group consisting of: I, V and T;
wherein any of the non-variable amino acids may be replaced with a conservative substitution.
Statement 47: The engineered desaturase of statement 46, wherein one or more of the variable amino acids within a motif is deleted.
Statement 48: The engineered desaturase of statement 46, wherein there is one or more amino acid insertions in any one of said motifs.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein. The invention can be more fully understood from the following description of the figure(s):

FIG. 1: Diagrammatic representation of the preferred conversion pathway of the native delta-15 desaturase and the preferred pathway of the engineered desaturase of the present invention

FIG. 2: multiple sequence alignment highlighting the regions of high diversity within the domains of delta-15 desaturases

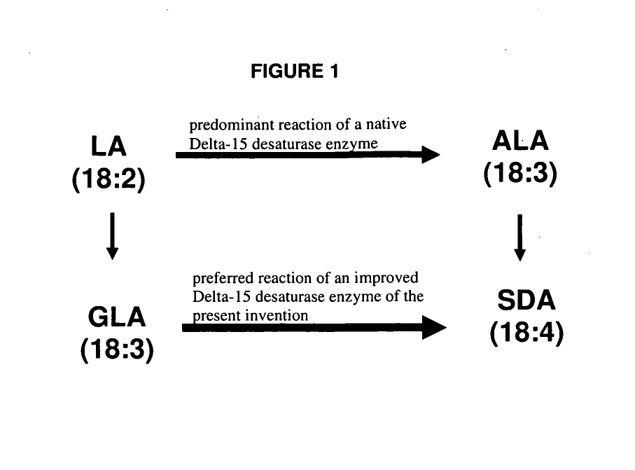

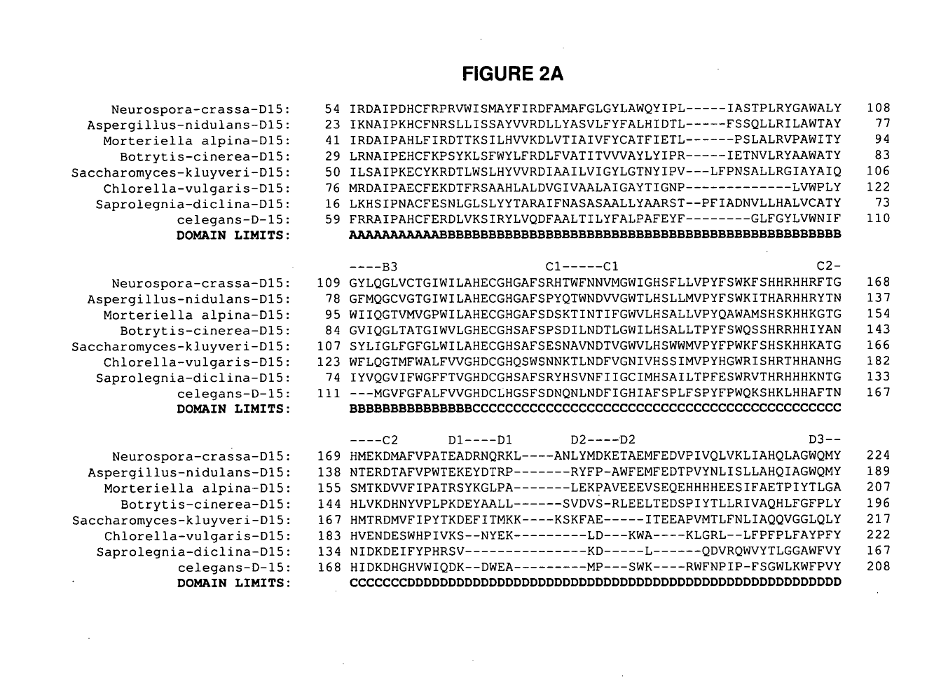

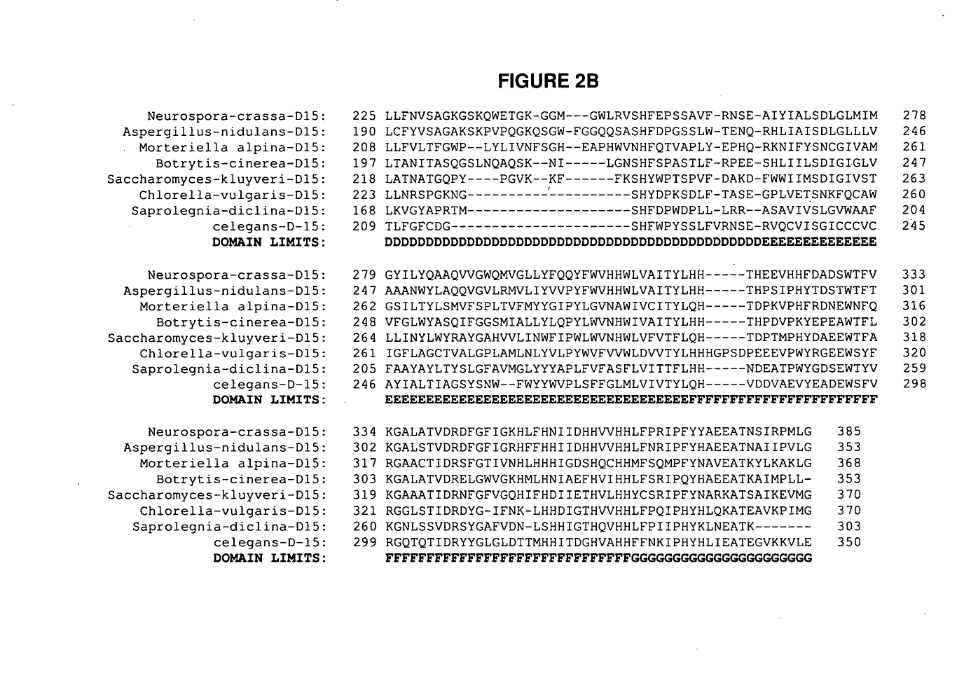

### DETAILED DESCRIPTION OF THE INVENTION

The present invention overcomes the limitations of the prior art by providing methods and compositions for creation of plants with improved PUFA content. The modification of fatty acid content of an organism such as a plant presents many advantages, including improved nutrition and health benefits. Modification of fatty acid content can be used to achieve beneficial levels or profiles of desired PUFAs in plants, plant parts, and plant products, including plant seed oils. For example, when the desired PUFAs are produced in the seed tissue of a plant, the oil may be isolated from the seeds typically resulting in an oil high in desired PUFAs or an oil having a desired fatty acid content or profile, which may in turn be used to provide beneficial characteristics in food stuffs and other products. The invention in particular provides endogenous oils having SDA while also containing a beneficial oleic acid content.

Various aspects of the present invention include methods and compositions for modification of PUFA content of a cell, for example, modification of the PUFA content of a plant cell(s). Compositions related to the invention include novel isolated polynucleotide sequences, polynucleotide constructs and plants and/or plant parts transformed by polynucleotides of the invention. The isolated polynucleotide may encode a fatty acid desaturase and, in particular, may encode an engineered Δ15-desaturase. Host cells may be manipulated to express a polynucleotide encoding a desaturase polypeptide(s) that catalyzes desaturation of a fatty acid(s).

Some aspects of the present invention include various desaturase polypeptides and polynucleotides encoding the same. Various embodiments of the invention may use different combinations of desaturase polynucleotides and the encoded polypeptides, depending upon the host cell, the availability of substrate(s), and the desired end product(s).

### Polynucleotide Molecules, Polypeptide Molecules, Motifs, Fragments, Chimeric Molecules

The following definitions and methods are provided to better define the present invention and to guide those of ordinary skill in the art in the practice of the present invention. Unless otherwise noted, terms are to be understood according to conventional usage by those of ordinary skill in the relevant art.

As used herein, the term “polynucleotide molecule” refers to the single- or double-stranded DNA or RNA molecule of genomic or synthetic origin, i.e., a polymer of deoxyribonucleotide or ribonucleotide bases, respectively, read from the 5′ (upstream) end to the 3′ (downstream) end. As used herein, the term “polynucleotide sequence” refers to the sequence of a polynucleotide molecule. The nomenclature for nucleotide bases as set forth at 37 CFR § 1.822 is used herein.

The term “polypeptide” refers to any chain of amino acids, regardless of length or post-translational modification (e.g., glycosylation or phosphorylation). Considerations for choosing a specific polypeptide having desaturase activity include, but are not limited to, the pH optimum of the polypeptide, whether the polypeptide is a rate limiting enzyme or a component thereof, whether the desaturase used is essential for synthesis of a desired PUFA, and/or a co-factor is required by the polypeptide. The expressed polypeptide may have characteristics that are compatible with the biochemical environment of its location in the host cell. For example, the polypeptide may have to compete for substrate(s).

“Desaturase” refers to a polypeptide that can desaturate, or catalyze formation of a double bond between, consecutive carbons of one or more fatty acids to produce a mono- or poly-unsaturated fatty acid or precursor thereof. Of particular interest are polypeptides that can catalyze the conversion of stearic acid to oleic acid, oleic acid to LA, LA to ALA, or GLA to SDA, which includes enzymes that desaturate at the 12, 15, or 6 positions. Preferred desaturases of the present invention include those that desaturate at the 15 position of the fatty acid chain.

As used herein, the term “fragment” or “fragment thereof” refers to a finite polynucleotide sequence length that comprises at least 25, at least 50, at least 75, at least 85, or at least 95 contiguous nucleotide bases, wherein its complete sequence in entirety is identical to a contiguous component of the referenced polynucleotide molecule. The term “fragment” also references a finite polypeptide length that comprises at least 10, at least 25, at least 50, at least 75, at least 100 or at least 150 contiguous amino acids, wherein its complete sequence in entirety is identical to a contiguous component of the referenced polynucleotide molecule. The polypeptide fragment exhibits some level of desaturase activity.

As used herein, the term “chimeric” refers to the product of the fusion of portions of two or more different polynucleotide or polypeptide molecules. As used herein, the term “chimeric” refers to a desaturase molecule produced through the concatenation of polynucleotide molecules or polypeptide molecules of known desaturases or other polypeptide molecules, or any copies thereof.

The phrases “coding sequence,” “structural sequence,” and “transcribable polynucleotide sequence” refer to a physical structure comprising an orderly arrangement of nucleic acids. The nucleic acids are arranged in a series of nucleic acid triplets that each form a codon. Each codon encodes for a specific amino acid. Thus the coding sequence, structural sequence, and transcribable polynucleotide sequence encode a series of amino acids forming a protein, polypeptide, or peptide sequence. The coding sequence, structural sequence, and transcribable polynucleotide sequence may be contained, without limitation, within a larger nucleic acid molecule, vector, etc. In addition, the orderly arrangement of nucleic acids in these sequences may be depicted, without limitation, in the form of a sequence listing, figure, table, electronic medium, etc.

As used herein, the term “parent” or “parental” refers to a molecule or a set of molecules that are analyzed for desired properties and from which novel, engineered molecules may be designed.

The term “engineered” refers to any polynucleotide or polypeptide molecule that has been created from manipulation of at least one parental sequence, such that the resultant molecular sequence is not identical to that of the parental molecule. Such a resultant molecule that is engineered from a parental molecule is referred to as a “variant”.

As used herein, the term “operably linked” refers to a first polynucleotide molecule, such as a promoter, connected with a second transcribable polynucleotide molecule, such as a gene of interest, where the polynucleotide molecules are so arranged that the first polynucleotide molecule affects the function of the second polynucleotide molecule. The two polynucleotide molecules may or may not be part of a single contiguous polynucleotide molecule and may or may not be adjacent. For example, a promoter is operably linked to a gene of interest if the promoter regulates or mediates transcription of the gene of interest in a cell.

The invention disclosed herein provides for polypeptide molecules that exhibit delta-15 desaturase enzymatic activity, and methods for producing and using the same.

### Polynucleotide Isolation and Modification

Any number of methods well known to those skilled in the art can be used to isolate a polynucleotide molecule, or fragment thereof, disclosed in the present invention. For example, PCR (polymerase chain reaction) technology can be used to amplify flanking regions from a genomic library of a plant using publicly available sequence information. A number of methods are known to those of skill in the art to amplify unknown polynucleotide molecules adjacent to a core region of known polynucleotide sequence. Methods include but are not limited to inverse PCR (IPCR), vectorette PCR, Y-shaped PCR, and genome walking approaches. Polynucleotide fragments can also be obtained by other techniques such as by directly synthesizing the fragment by chemical means, as is commonly practiced by using an automated oligonucleotide synthesizer.

As used herein, the term “isolated polynucleotide molecule” refers to a polynucleotide molecule at least partially separated from other molecules normally associated with it in its native state. In one embodiment, the term “isolated” is also used herein in reference to a polynucleotide molecule that is at least partially separated from nucleic acids that normally flank the polynucleotide in its native state. Thus, polynucleotides fused to regulatory or coding sequences with which they are not normally associated, for example as the result of recombinant techniques, are considered isolated herein. Such molecules are considered isolated even when present, for example in the chromosome of a host cell, or in a nucleic acid solution. The term “isolated” as used herein is intended to encompass molecules not present in their native state.

Those of skill in the art are familiar with the standard resource materials that describe specific conditions and procedures for the constriction, manipulation, and isolation of macromolecules (e.g., polynucleotide molecules, plasmids, etc.), as well as the generation of recombinant organisms and the screening and isolation of polynucleotide molecules.

Short nucleic acid sequences having the ability to specifically hybridize to complementary nucleic acid sequences may be produced and utilized in the present invention. These short nucleic acid molecules may be used as probes to identify the presence of a complementary nucleic acid sequence in a given sample. Thus, by constructing a nucleic acid probe that is complementary to a small portion of a particular nucleic acid sequence, the presence of that nucleic acid sequence may be detected and assessed. Use of these probes may greatly facilitate the identification of transgenic plants that contain the presently disclosed nucleic acid molecules. The probes may also be used to screen cDNA or genomic libraries for additional nucleic acid sequences related or sharing homology to the presently disclosed promoters and transcribable polynucleotide sequences. The short nucleic acid sequences may be used as probes and specifically as PCR probes. A PCR probe is a nucleic acid molecule capable of initiating a polymerase activity while in a double-stranded structure with another nucleic acid. Various methods for determining the structure of PCR probes and PCR techniques exist in the art. Computer generated searches using programs such as Primer3, STSPipeline, or GeneUp (Pesole, et al., 1998), for example, can be used to identify potential PCR primers.

Alternatively, the short nucleic acid sequences may be used as oligonucleotide primers to amplify or mutate a complementary nucleic acid sequence using PCR technology. These primers may also facilitate the amplification of related complementary nucleic acid sequences (e.g. related nucleic acid sequences from other species).

The primer or probe is generally complementary to a portion of a nucleic acid sequence that is to be identified, amplified, or mutated. The primer or probe should be of sufficient length to form a stable and sequence-specific duplex molecule with its complement. The primer or probe in some embodiments is about 10 to about 200 nucleotides long, in some embodiments is about 10 to about 100 nucleotides long, in some embodiments is about 10 to about 50 nucleotides long, and in some embodiments is about 14 to about 30 nucleotides long. The primer or probe may be prepared by direct chemical synthesis, by PCR (See, for example, U.S. Pat. Nos. 4,683,195, and 4,683,202, each of which is herein incorporated by reference), or by excising the nucleic acid specific fragment from a larger nucleic acid molecule.

The term “recombinant vector” as used herein, includes any recombinant segment of DNA that one desires to introduce into a host cell, tissue and/or organism, and specifically includes expression cassettes isolated from a starting polynucleotide. A recombinant vector may be linear or circular. In various aspects, a recombinant vector may comprise at least one additional sequence chosen from the group consisting of: regulatory sequences operatively linked to the polynucleotide; selection markers operatively coupled to the polynucleotide; marker sequences operatively coupled to the polynucleotide; a purification moiety operatively coupled to the polynucleotide; and a targeting sequence operatively coupled to the polynucleotide.

### Modifications and Engineering of the Desaturase Molecular Sequence

A number of enzymes are involved in PUFA biosynthesis. LA, (18:2, Δ9, 12) is produced from oleic acid (OA, 18:1, Δ9) by a Δ12-desaturase, while ALA (18:3) is produced from LA by a Δ15-desaturase. SDA (18:4, Δ6, 9, 12, 15) and GLA (18:3, Δ6, 9, 12) are produced from LA and ALA by a Δ6-desaturase. However, as stated above, mammals cannot desaturate beyond the Δ9 position and therefore cannot convert oleic acid into LA. Likewise, ALA cannot be synthesized by mammals. Other eukaryotes, including fungi and plants, have enzymes that desaturate at the carbon 12 and carbon 15 position. The major poly unsaturated fatty acids of animals therefore are derived from diet via the subsequent desaturation and elongation of dietary LA and ALA.

U.S. Pat. No. 5,952,544 (herein incorporated by reference in its entirety) describes nucleic acid fragments isolated and cloned from Brassica napus that encode fatty acid desaturase enzymes. Expression of these fragments in plants results in accumulation of ALA. However, these plants also accumulate LA, which remains unconverted by the desaturase. An enzyme that converts more LA to ALA would be advantageous. Increased conversion from LA to ALA would create greater amounts of ALA. Increased ALA levels allow the Δ6-desaturase, when co-expressed with a nucleic acid encoding for the Δ15-desaturase, to act upon the ALA, thereby producing greater levels of SDA. Because of the multitude of beneficial uses for SDA, the present invention encompasses the recognition that it would be desirable to create a substantial increase in the yield of SDA. Nucleic acids from various sources have been sought to increase SDA yield. Further, innovations that would allow for improved commercial production in land-based crops are still highly desired. (See, e.g., Reed et al., 2000).

Fatty acid desaturases are enzymes that introduce double bonds into fatty acyl chains. They are present in all groups of organisms, i.e., bacteria, fungi, plants and animals, and play a key role in the maintenance of the proper structure and functioning of biological membranes. With the exception of the stearoyl-ACP desaturase and its relatives from plants, fatty acids desaturases are integral membrane proteins, believed to contain two iron atoms in their active site. All known desaturases are characterized by the presence of three histidine clusters, which are localized at strongly conserved positions in the amino acid sequence of each protein. It has been suggested that these clusters might be involved in the formation of the active site of each desaturase, as has been demonstrated in other di-iron enzymes. It is assumed in the current scientific literature that the histidine clusters and iron ions constitute the catalytic centre of the desaturase, although other regions of the protein have been shown to impact enzymatic activity.

Nucleic acids encoding Δ15-desaturases have been isolated from several species of cyanobacteria, fungi (including Saccharomyces, Botrytis, Chlorella, Aspergillus, Mortierella and Neurospora) and plants (including Arabidopsis, soybean, and parsley). Structural models of the protein family have been generated for several species (Diaz et al. 2002; Knipple et al., 2002; Sasata et al., 2004; Sperling et al., 2003). Proteins that are known desaturases share the common PFAM domain PF00487. Key structural features of desaturases localized to the endoplasmic reticulum membrane include: two membrane anchor regions (domains B and E), each consisting of two transmembrane domains; a presumed active site formed by the interaction of domains C, D, F, and G; three histidine residues extended away from the cytosolic face of the membrane that coordinate binding of iron, which plays a role in catalysis; and probable presentation of the substrate to the enzyme in the membrane (as opposed to from the cytosol). The deduced amino acid sequences of these desaturases demonstrate some degree of similarity, most notably in the region of three histidine-rich motifs that, without being bound by any one theory, are believed to be involved in iron binding. Delta-15 desaturases desaturate both LA to produce ALA, and GLA to produce SDA. The known native enzymes either prefer LA as a substrate over GLA, or do not exhibit a preference for either substrate. The present invention includes and provides delta-15 desaturases that exhibit both increased enzymatic activity and improved substrate selectivity of GLA vs. LA, as compared to the native wild-type enzyme.

If desired, the regions of a desaturase polypeptide important for desaturase activity may be manipulated through means such as gene engineering or routine mutagenesis followed by expression of the resulting mutant polypeptides and determination of their activities. Mutants may include substitutions, deletions, insertions and point mutations, combinations thereof, or other types of sequence manipulations. Substitutions may be made on the basis of conserved hydrophobicity or hydrophilicity (Kyte and Doolittle, 1982), or on the basis of the ability to assume similar polypeptide secondary structure (Chou and Fasman. 1978). A typical functional analysis begins with deletion mutagenesis to determine the N- and C-terminal limits of the protein necessary for function, and then internal deletions, insertions or point mutants are made to further determine regions necessary for function. Other techniques such as cassette mutagenesis or total synthesis also can be used. Deletion mutagenesis is accomplished, for example, by using exonucleases to sequentially remove the 5′ or 3′ coding regions. Kits are available for such techniques. After deletion, the coding region is completed by ligating oligonucleotides containing start or stop codons to the deleted coding region after 5′ or 3′ deletion, respectively. Alternatively, oligonucleotides encoding start or stop codons are inserted into the coding region by a variety of methods including site-directed mutagenesis, mutagenic PCR or by ligation onto DNA digested at existing restriction sites.

Internal deletions can similarly be made through a variety of methods including the use of existing restriction sites in the DNA, by use of mutagenic primers via site directed mutagenesis or mutagenic PCR. Insertions are made through methods such as linker-scanning mutagenesis, site-directed mutagenesis or mutagenic PCR. Point mutations are made through techniques such as site-directed mutagenesis or mutagenic PCR. Chemical mutagenesis may also be used for identifying regions of a desaturase polypeptide important for activity. Such structure-function analysis can determine which regions may be deleted, which regions tolerate insertions, and which point mutations allow the mutant protein to function in substantially the same way as the native desaturase. All such mutant proteins and nucleotide sequences encoding them are within the scope of the present invention.

Desaturase molecules may be engineered or designed to optimize a particular phenotype. For example, a delta-15 desaturase gene may be engineered to provide an increased expression level of a product in a host cell or organism, to preferentially interact with one substrate molecule over another, or to exhibit an altered kinetic profile.

The term “engineered” refers to any polynucleotide or polypeptide molecule that has been created from manipulation of at least one parental sequence, such that the resultant molecular sequence is not identical to that of the parental molecule. Various techniques for effecting such changes are known in the art. For example, such molecules may be generated by interchanging one or more amino acids identified from one desaturase with those identified from a different desaturase. Another example would be the introduction of conservative or non-conservative amino acid changes to the native parent molecule. Yet another example would be the creation of a chimeric molecule comprising fragments of sequences from different parental molecules. Engineered delta-15 desaturases of the present invention include those generated by the manipulation of regions identified from a parental fungal delta-15 desaturase, in particular a delta-15 desaturase from Mortierella alpina. It is contemplated that delta-15 desaturase molecules identified from other organisms could also be used to engineer variants that exhibit a particular desired phenotype or activity.

Thus, the design and production of delta-15 desaturases that exhibit an improved phenotype over known wild-type desaturases is one aspect of the present invention. Preferred embodiments include delta-15 desaturases that prefer the substrate GLA over LA, thereby producing SDA in preference to ALA.

The molecules disclosed in the present invention are illustrative of such engineered delta-15 desaturases. Briefly, sequence alignments of delta-15 desaturases from various sources, including Mortierella alpina, Neurospora crassa, Saccharomyces kluyveri, Aspergillus nidulans and Chlorella vulgaris, revealed regions of the molecules that comprise highly variable amino acid sequences in addition to more conserved regions. The regions of high diversity were selected for molecular engineering experiments for the purpose of generating molecules with novel characteristics, such as substrate preference and/or enzymatic activity. Using the Mortierella alpina delta-15 desaturase protein as a parent protein, changes were designed in these highly variable regions to sample from the diversity observed in naturally occurring delta-15 desaturases. Additional conservative amino acid substitutions were included in the designs as well. Polynucleotide sequences were then engineered to correspond to the amino acid variants designed from the bioinformatics analysis.

### Enzyme Activity and Kinetics

Analyses of the Kₘ (Michaelis constant) and specific activity of a polypeptide in question may be considered in determining the suitability of a given polypeptide for modifying PUFA(s) production, level, or profile in a given host cell. The polypeptide used in a particular situation is one that typically can function under the conditions present in the intended host cell, but otherwise may be any desaturase polypeptide having a desired characteristic or being capable of modifying the relative production, level or profile of a desired PUFA(s) or any other desired characteristics as discussed herein. The substrate(s) for the expressed enzyme may be produced by the host cell or may be exogenously supplied. To achieve expression, the polypeptide(s) of the instant invention are encoded by polynucleotides as described below.

The inventors have engineered enzymes from parental fungal enzymes. Fungal sources can include, but are not limited to, the genus Aspergillus, e.g., Aspergillus nidulans; the genus Botrytis, e.g., Botrytis cinerea; the genus Neurospora, e.g., Neurospora crassa; the genus Mortierella, e.g. Mortierella alpina; and other fungi that exhibit Δ15-desaturase activity.

The polynucleotide molecules encoding the engineered Δ15-desaturase may be expressed in transgenic plants, microorganisms or animals to effect greater synthesis of SDA from GLA. Other polynucleotides that are substantially identical to the disclosed Δ15-desaturase polynucleotides, or that encode polypeptides that are substantially identical to the disclosed Δ15-desaturase polypeptide, may also be used.

Encompassed by the present invention are molecules engineered from at least one known desaturase. Such known desaturases include variants of the disclosed Δ15-desaturases, or desaturases naturally occurring within a species of fungus. Desaturases may be identified by their ability to catalyze the formation of a double bond between two consecutive amino acids of a fatty acid chain. Desaturases may also be identified by screening sequence databases for sequences homologous to the disclosed desaturases, by hybridization of a probe based on the disclosed desaturases to a library constructed from the source organism, or by RT-PCR using mRNA from the source organism and primers based on the disclosed desaturases. Desaturase activity may further be elucidated by screening host organisms for production of said desaturase, or by screening the host organism for the product of the desaturase.

Certain aspects of the invention include variants and fragments of engineered Δ15-desaturase polypeptides that exhibit desaturase activity, and the nucleic acids encoding such. In another aspect of the invention, a vector comprising a nucleic acid, or fragment thereof, comprising a promoter, a Δ15-desaturase coding sequence and a termination region may be transferred into an organism in which the promoter and termination regions are functional. Accordingly, organisms producing an engineered Δ15-desaturase are provided by this invention. Yet another aspect of this invention provides an isolated, engineered Δ15-desaturase that can be purified from the recombinant organisms by standard methods of protein purification. (For example, see Ausubel et al., 1987).

### Determination of Sequence Similarity Using Hybridization Techniques

Nucleic acid hybridization is a technique well known to those of skill in the art of DNA manipulation. The hybridization properties of a given pair of nucleic acids are an indication of their similarity or identity.

The term “hybridization” refers generally to the ability of nucleic acid molecules to join via complementary base strand pairing. Such hybridization may occur when nucleic acid molecules are contacted under appropriate conditions. “Specifically hybridizes” refers to the ability of two nucleic acid molecules to form an anti-parallel, double-stranded nucleic acid structure. A nucleic acid molecule is said to be the “complement” of another nucleic acid molecule if they exhibit “complete complementarity,” i.e., each nucleotide in one sequence is complementary to its base pairing partner nucleotide in another sequence. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Nucleic acid molecules that hybridize to other nucleic acid molecules, e.g., at least under low stringency conditions, are said to be “hybridizable cognates” of the other nucleic acid molecules. Conventional low stringency and high stringency conditions are described herein and by Sambrook et al., Molecular Cloning, A Laboratory Manual, 2nd Ed., Cold Spring Harbor Press, Cold Spring Harbor, N.Y. (1989) and by Haymes et al., 1985. Departures from complete complementarity are permissible, as long as such departures do not completely preclude the capacity of the molecules to form a double-stranded structure.

Low stringency conditions may be used to select nucleic acid sequences with lower sequence identities to a target nucleic acid sequence. One may wish to employ conditions such as about 0.15 M to about 0.9 M sodium chloride, at temperatures ranging from about 20° C. to about 55° C. High stringency conditions may be used to select for nucleic acid sequences with higher degrees of identity to the disclosed nucleic acid sequences (Sambrook et al., 1989). High stringency conditions typically involve nucleic acid hybridization in about 2× to about 10×SSC (diluted from a 20×SSC stock solution containing 3 M sodium chloride and 0.3 M sodium citrate, pH 7.0 in distilled water), about 2.5× to about 5×Denhardt's solution (diluted from a 50× stock solution containing 1% (w/v) bovine serum albumin, 1% (w/v) ficoll, and 1% (w/v) polyvinylpyrrolidone in distilled water), about 10 mg/mL to about 100 mg/mL fish sperm DNA, and about 0.02% (w/v) to about 0.1% (w/v) SDS, with an incubation at about 50° C. to about 70° C. for several hours to overnight. High stringency conditions may be provided by 6×SSC, 5×Denhardt's solution, 100 mg/mL fish sperm DNA, and 0.1% (w/v) SDS, with an incubation at 55° C. for several hours. Hybridization is generally followed by several wash steps. The wash compositions generally comprise 0.5× to about 10×SSC, and 0.01% (w/v) to about 0.5% (w/v) SDS with a 15 minute incubation at about 20° C. to about 70° C. In one embodiment, the nucleic acid segments remain hybridized after washing at least one time in 0.1×SSC at 65° C.

A nucleic acid molecule in one embodiment of the present invention comprises a nucleic acid sequence that hybridizes, under low or high stringency conditions, with SEQ ID NO: 332 through SEQ ID NO: 662, any complements thereof, or any fragments thereof, or any cis elements thereof. A nucleic acid molecule in one embodiment of the present invention comprises a nucleic acid sequence that hybridizes under high stringency conditions with SEQ ID NO: 332 through SEQ ID NO: 662, any complements thereof, or any fragments thereof, or any cis elements thereof.

### Analysis of Sequence Similarity Using Identity Scoring

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components that are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT. FASTA, and TFASTA available as part of the GCG® Wisconsin Package® (Accelrys Inc., San Diego, Calif.). An “identity fraction” for aligned segments of a test sequence and a reference sequence is the number of identical components that are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences. Each of the aforementioned algorithms is well known in the art.

As used herein, the term “substantial percent sequence identity” refers to a percent sequence identity of at least about 70% sequence identity, at least about 80% sequence identity, at least about 85% identity, at least about 90% sequence identity, or even greater sequence identity, such as about 95%, 96%, 97% 98% or about 99% sequence identity with a molecular sequence described herein. Molecules that provide delta-15 desaturase activity and have a substantial percent sequence identity to the molecules provided herein are encompassed within the scope of this invention. “Substantially identical” refers to an amino acid sequence or nucleic acid sequence exhibiting at least 70%, 80%, 85%, 90% or 95% or even greater identity such as 96%, 97%, 98%, or 99% identity to the parental Δ15-desaturase amino acid sequence or nucleic acid sequence encoding the amino acid sequence. Polypeptide or polynucleotide comparisons may be carried out using sequence analysis software, for example, the Sequence Analysis software package of the GCG Wisconsin Package (Accelrys, San Diego, Calif.), MEGAlign (DNAStar, Inc., 1228 S. Park St., Madison, Wis. 53715), and MacVector (Oxford Molecular Group, 2105 S. Bascom Avenue, Suite 200, Campbell, Calif. 95008). Such software matches similar sequences by assigning degrees of similarity or identity.

“Homology” refers to the level of similarity between two or more nucleic acid or amino acid sequences in terms of percent of positional identity (i.e., sequence similarity or identity). Homology also refers to the concept of similar functional properties among different nucleic acids or proteins.

For purposes of this invention “percent identity” may also be determined using BLASTX version 2.0 for translated nucleotide sequences and BLASTN version 2.0 for polynucleotide sequences. In a preferred embodiment of the present invention, the presently disclosed delta-15 desaturase molecules comprise nucleic acid molecules or fragments having a BLAST score of more than 200, preferably a BLAST score of more than 300, and even more preferably a BLAST score of more than 400 with their respective homologues.

### Regulatory Elements Controlling the Expression of the Desaturase Gene

Regulatory elements, such as promoters, play a pivotal role in enhancing the agronomic, pharmaceutical or nutritional value of crops. Examples of promoters include constitutive promoters such as those disclosed in U.S. Pat. No. 5,641,876 (rice actin promoter, herein incorporated by reference in its entirety) U.S. Pat. No. 6,177,611 (constitutive maize promoters, herein incorporated by reference in its entirety), U.S. Pat. Nos. 5,322,938, 5,352,605, 5,359,142 and 5,530,196, all of which are herein incorporated by reference in their entireties (35S promoter); specific promoters such as those disclosed in U.S. Pat. No. 6,433,252 (maize L3 oleosin promoter, P-Zm.L3, herein incorporated by reference in its entirety), U.S. Pat. No. 5,837,848 (root specific promoter, herein incorporated by reference in its entirety), U.S. Pat. No. 6,294,714 (light inducible promoters, herein incorporated by reference in its entirety), U.S. Pat. No. 6,140,078 (salt inducible promoters, herein incorporated by reference in its entirety), U.S. Pat. No. 6,252,138 (pathogen inducible promoters, herein incorporated by reference in its entirety), U.S. Pat. No. 6,175,060 herein incorporated by reference in its entirety (phosphorus deficiency inducible promoters), U.S. Pat. No. 6,635,806 herein incorporated by reference in its entirety (gama-coixin promoter, P-Cl.Gcx), and U.S. patent application Ser. No. 09/757,089 herein incorporated by reference in its entirety (maize chloroplast aldolase promoter), all of which are incorporated herein by reference in their entirety. Examples of useful tissue-specific, developmentally-regulated promoters include the β-conglycinin 7Sα promoter (Doyle et al., 1986; Tierney et al., 1987), and seed-specific promoters (Knutzon, et al., 1992; Bustos, et al., 1991; Lam and Chua, 1991). Plant functional promoters useful for preferential expression in seed plastid include those from plant storage proteins and from proteins involved in fatty acid biosynthesis in oilseeds. Examples of such promoters include the 5′ regulatory regions from such transcribable polynucleotide sequences as napin (Kridl et al., 1991), phaseolin, zein, soybean trypsin inhibitor, ACP, stearoyl-ACP desaturase, and oleosin. Seed-specific regulation is discussed in EP 0 255 378 (herein incorporated by reference in its entirety). Another exemplary tissue-specific promoter is the lectin promoter, which is specific for seed tissue. The lectin protein in soybean seeds is encoded by a single transcribable polynucleotide sequence (Le1) that is only expressed during seed maturation and accounts for about 2 to about 5% of total seed mRNA. The lectin transcribable polynucleotide sequence and seed-specific promoter have been fully characterized and used to direct seed specific expression in transgenic tobacco plants (Vodkin, et al., 1983; Lindstrom, et al., 1990).

Polynucleotides encoding desaturases may be placed under transcriptional control of a promoter. In some cases this leads to an increase in the amount of desaturase enzyme expressed and concomitantly an increase in the fatty acid produced as a result of the reaction catalyzed by the enzyme. There is a wide variety of plant promoter sequences that may be used to drive tissue-specific expression of polynucleotides encoding desaturases in transgenic plants. For instance, the napin promoter and the acyl carrier protein promoters have previously been used in the modification of seed oil composition by expression of an antisense form of a desaturase (Knutzon et al. 1999). Similarly, the promoter for the β-subunit of soybean β-conglycinin has been shown to be highly active and to result in tissue-specific expression in transgenic plants of species other than soybean (Bray et al., 2004). Arondel et al. (1992) increased the amount of linolenic acid (18:3) in tissues of transgenic Arabidopsis plants by placing the endoplasmic reticulum-localized fad3 gene under transcriptional control of the strong constitutive cauliflower mosaic virus 35S promoter.

### Constructs and Vectors

Nucleic acid constructs may be provided that integrate into the genome of a host cell or are autonomously replicated (e.g., episomally replicated) in the host cell. For production of ALA and/or SDA, the expression cassettes, (i.e., a polynucleotide encoding a protein that is operatively linked to nucleic acid sequence(s) that directs the expression of the polynucleotide) generally used include an expression cassette that provides for expression of a polynucleotide encoding a Δ15-desaturase. In certain embodiments a host cell may have wild type fatty acid content.

As used herein, the term “construct” means any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked.

As used herein, the term “vector” means any recombinant polynucleotide construct that may be used for the purpose of transformation, i.e. the introduction of heterologous DNA into a host cell. Vectors used for plant transformation may include, for example, plasmids, cosmids, YACs (yeast artificial chromosomes), BACs (bacterial artificial chromosomes) or any other suitable cloning system, as well as fragments of DNA therefrom. Thus when the term “vector” or “expression vector” is used, all of the foregoing types of vectors, as well as nucleic acid sequences isolated therefrom, are included. It is contemplated that utilization of cloning systems with large insert capacities will allow introduction of large DNA sequences comprising more than one selected gene. In accordance with the invention, this could be used to introduce various desaturase encoding nucleic acids. Introduction of such sequences may be facilitated by use of bacterial or yeast artificial chromosomes (BACs or YACs, respectively), or even plant artificial chromosomes. For example, the use of BACs for Agrobacterium-mediated transformation was disclosed by Hamilton et al. (1996).

Particularly useful for transformation are expression cassettes that have been isolated from such vectors. DNA segments used for transforming plant cells will, of course, generally comprise the cDNA, gene or genes which one desires to introduce into and have expressed in the host cells. These DNA segments can further include structures such as promoters, enhancers, polylinkers, or even regulatory genes as desired. The DNA segment or gene chosen for cellular introduction will often encode a protein that will be expressed in the resultant recombinant cells resulting in a screenable or selectable trait and/or that will impart an improved phenotype to the resulting transgenic plant. However, this may not always be the case, and the present invention also encompasses transgenic plants incorporating non-expressed transgenes.

Methods and compositions for the construction of expression vectors, when taken in light of the teachings provided herein, for expression of desaturase enzymes will be apparent to one of ordinary skill in the art. Expression vectors, as described herein, are DNA or RNA molecules engineered for controlled expression of a desired polynucleotide, e.g., the Δ15-desaturase encoding polynucleotide. Examples of vectors include plasmids, bacteriophages, cosmids or viruses. Shuttle vectors, e.g. (Wolk et al. 1984; Bustos et al., 1991) are also contemplated in accordance with the present invention. Reviews of vectors and methods of preparing and using them can be found in Sambrook et al. (1989) and Goeddel (1990). Sequence elements capable of effecting expression of a polynucleotide include promoters, enhancer elements, upstream activating sequences, transcription termination signals and polyadenylation sites.

The constructs of the present invention may be any commercially-available expression vector, including the pYES2.1 or a double Ti plasmid border DNA constructs. Such Ti plasmid constructs have the right border (RB or AGRtu.RB) and left border (LB or AGRtu.LB) regions of the Ti plasmid isolated from Agrobacterium tumefaciens comprising a T-DNA, that along with transfer molecules provided by the Agrobacterium cells, permit the integration of the T-DNA into the genome of a plant cell (see for example U.S. Pat. No. 6,603,061, herein incorporated by reference in its entirety). The constructs may also comprise the plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often Agrobacterium tumefaciens ABI, C58, or LBA4404, however, other strains known to those skilled in the art of plant transformation can function in the present invention.

Methods are known in the art for assembling and introducing constructs into a cell in such a manner that the transcribable polynucleotide molecule is transcribed into a functional mRNA molecule that is translated and expressed as a protein product. For the practice of the present invention, conventional compositions and methods for preparing and using constructs and host cells are well known to one skilled in the art, see for example, Molecular Cloning: A Laboratory Manual, 3rd edition Volumes 1, 2, and 3 (2000) J. F. Sambrook, D. W. Russell, and N. Irwin, Cold Spring Harbor Laboratory Press. Methods for making recombinant vectors particularly suited to plant transformation include, without limitation, those described in U.S. Pat. Nos. 4,971,908, 4,940,835, 4,769,061 and 4,757,011, all of which are herein incorporated by reference in their entireties.

Thus, one embodiment of the present invention is a construct comprising a regulatory element operably linked to a transcribable polynucleotide molecule as provided in SEQ ID NO: 332 through SEQ ID NO: 662 so as to modulate transcription of said transcribable polynucleotide molecule at a desired level or in a desired tissue or developmental pattern upon introduction of said construct into a plant cell. Modifications of the nucleotide sequences disclosed herein that maintain the functions contemplated herein are within the scope of this invention. Such modifications may include insertions, substitutions and deletions, and specifically substitutions which reflect the degeneracy of the genetic code.

As an example, a vector appropriate for expression of a Δ15-desaturase in transgenic plants can comprise a seed-specific promoter sequence derived from helianthinin, napin, or glycinin operably linked to the Δ15-desaturase coding region and further operably linked to a seed storage protein termination signal or the nopaline synthase termination signal. As a still further example, a vector for use in expression of Δ15-desaturase in plants can comprise a constitutive promoter or a tissue specific promoter operably linked to the Δ15-desaturase coding region and further operably linked to a constitutive or tissue specific terminator or the nopaline synthase termination signal.

In certain embodiments, the expression cassettes may include a cassette that provides for Δ6- and/or Δ15-desaturase activity, particularly in a host cell that produces or can take up LA or ALA, respectively. The host ALA production can be removed, reduced and/or inhibited by inhibiting the activity of the endogenous Δ15-desaturase. This can be accomplished by standard selection, by providing an expression cassette for an antisense Δ15-desaturase, by disrupting a target Δ15-desaturase gene through insertion, deletion, substitution of part or all of the target gene, or by adding an inhibitor of Δ15-desaturase. Production of omega-6 type unsaturated fatty acids, such as LA, is favored in a host organism that is incapable of producing ALA. Similarly, production of LA or ALA is favored in a microorganism or animal having Δ6-desaturase activity by providing an expression cassette for an antisense Δ6 transcript, by disrupting a Δ6-desaturase gene, or by use of a Δ6-desaturase inhibitor.

Polynucleotides encoding desired desaturases can be identified in a variety of ways. As an example, a source of the desired desaturase, for example genomic or cDNA libraries, is screened with detectable enzymatically- or chemically-synthesized probes, which can be made from DNA, RNA, or non-naturally occurring nucleotides, or mixtures thereof. Probes may be enzymatically synthesized from polynucleotides of known desaturases for normal or reduced-stringency hybridization methods. Oligonucleotide probes also can be used to screen sources and can be based on sequences of known desaturases, including sequences conserved among known desaturases, or on peptide sequences obtained from the desired purified protein. Oligonucleotide probes based on amino acid sequences can be degenerate to encompass the degeneracy of the genetic code, or can be biased in favor of the preferred codons of the source organism. Oligonucleotides also can be used as primers for PCR from reverse transcribed mRNA from a known or suspected source; the PCR product can be the full length cDNA or can be used to generate a probe to obtain the desired full length cDNA. Alternatively, a desired protein can be entirely sequenced and total synthesis of a DNA encoding that polypeptide performed.

Some or all of the coding sequence for a polypeptide having desaturase activity may be from a natural source. In some situations, however, it is desirable to modify all or a portion of the codons, for example, to enhance expression, by employing host preferred codons. Host preferred codons can be determined from the codons of highest frequency in the proteins expressed in the largest amount in a particular host species of interest. Thus, the coding sequence for a polypeptide having desaturase activity can be synthesized in whole or in part. All or portions of the DNA also can be synthesized to remove any destabilizing sequences or regions of secondary structure which would be present in the transcribed mRNA. All or portions of the DNA also can be synthesized to alter the base composition to one more preferable in the desired host cell. Methods for synthesizing sequences and bringing sequences together are well established in the literature. In vitro mutagenesis and selection, site-directed mutagenesis, or other means can be employed to obtain mutations of naturally occurring desaturase genes to produce a polypeptide having desaturase activity in vivo with more desirable physical and kinetic parameters for function in the host cell, such as a longer half-life or a higher rate of production of a desired polyunsaturated fatty acid.

The choice of any additional elements used in conjunction with the desaturase coding sequences will often depend on the purpose of the transformation. One of the major purposes of transformation of crop plants is to add commercially desirable, agronomically important traits to the plant. As PUFAs are known to confer many beneficial effects on health, concomitant increases in SDA production may also be beneficial and could be achieved by expression of fungal Δ15-desaturase. Such increasing of SDA may, in certain embodiments of the invention, comprise expression of Δ6 and/or Δ12 desaturase, including fungal or plant Δ6 and/or Δ12 desaturases.

### Transformation

The term “transformation” refers to the introduction of nucleic acid into a recipient host. The term “host” refers to bacteria cells, fungi, animals and animal cells, plants and plant cells, or any plant parts or tissues including protoplasts, calli, roots, tubers, seeds, stems, leaves, seedlings, embryos, and pollen. As used herein, the term “transformed” refers to a cell, tissue, organ, or organism into which has been introduced a foreign polynucleotide molecule, such as a construct. The introduced polynucleotide molecule may be integrated into the genomic DNA of the recipient cell, tissue, organ, or organism such that the introduced polynucleotide molecule is inherited by subsequent progeny. A “transgenic” or “transformed” cell or organism also includes progeny of the cell or organism and progeny produced from a breeding program employing such a transgenic plant as a parent in a cross and exhibiting an altered phenotype resulting from the presence of a foreign polynucleotide molecule. The term “transgenic” refers to an animal, plant, or other organism containing one or more heterologous nucleic acid sequences.

Technology for introduction of DNA into cells is well known to those of skill in the art. The method generally comprises the steps of selecting a suitable host cell, transforming the host cell with a recombinant vector, and obtaining the transformed host cell. Expression in a host cell can be accomplished in a transient or stable fashion. Transient expression can occur from introduced constructs that contain expression signals functional in the host cell, but which constructs do not replicate and rarely integrate in the host cell, or where the host cell is not proliferating. Transient expression also can be accomplished by inducing the activity of a regulatable promoter operably linked to the gene of interest, although such inducible systems frequently exhibit a low basal level of expression. Stable expression can be achieved by introduction of a construct that can integrate into the host genome or that autonomously replicates in the host cell. Stable expression of the gene of interest can be selected for through the use of a selectable marker located on or transfected with the expression construct, followed by selection for cells expressing the marker. When stable expression results from integration, integration of constructs can occur randomly within the host genome or can be targeted through the use of constructs containing regions of homology with the host genome sufficient to target recombination with the host locus. Where constructs are targeted to an endogenous locus, all or some of the transcriptional and translational regulatory regions can be provided by the endogenous locus.

When increased expression of the desaturase polypeptide in the source organism is desired, several methods can be employed. Additional genes encoding the desaturase polypeptide can be introduced into the host organism. Expression from the native desaturase locus also can be increased through homologous recombination, for example by inserting a stronger promoter into the host genome to cause increased expression, by removing destabilizing sequences from either the mRNA or the encoded protein by deleting that information from the host genome, or by adding stabilizing sequences to the mRNA (U.S. Pat. No. 4,910,141, herein incorporated by reference in its entirety).

It is contemplated that more than one polynucleotide encoding a desaturase or a polynucleotide encoding more than one desaturase may be introduced and propagated in a host cell through the use of episomal or integrated expression vectors. Where two or more genes are expressed from separate replicating vectors, it is desirable that each vector has a different means of replication. Each introduced construct, whether integrated or not, should have a different means of selection. Judicious choices of regulatory regions, selection means and method of propagation of the introduced construct can be experimentally determined so that all introduced polynucleotides are expressed at the necessary levels to provide for synthesis of the desired products.

Of particular interest is the Δ15-desaturase-mediated production of PUFAs in eukaryotic host cells. Eukaryotic cells include plant cells, such as those from oil-producing crop plants, and other cells amenable to genetic manipulation, including fungal cells. The cells may be cultured or formed as part or all of a host organism including a plant. In a preferred embodiment, the host is a plant cell that produces and/or can assimilate exogenously supplied substrate(s) for a Δ15-desaturase, and preferably produces large amounts of one or more of the substrates.

The transformed host cell is grown under appropriate conditions adapted for a desired end result. For host cells grown in culture, the conditions are typically optimized to produce the greatest or most economical yield of PUFAs, which relates to the selected desaturase activity. Media conditions that may be optimized include: carbon source, nitrogen source, addition of substrate, final concentration of added substrate, form of substrate added, aerobic or anaerobic growth, growth temperature, inducing agent, induction temperature, growth phase at induction, growth phase at harvest, pH, density, and maintenance of selection.

### Transgenic Plants

The present invention further provides a method for providing transgenic plants with an increased content of ALA and/or SDA. This method includes, for example, introducing DNA encoding Δ15-desaturase into plant cells that lack or have low levels of ALA or SDA but contain LA, and regenerating plants with increased ALA and/or SDA content from the transgenic cells. In certain embodiments of the invention, a DNA encoding a Δ6- and/or Δ12-desaturase may also be introduced into the plant cells. Such plants may or may not also have endogenous Δ6- and/or Δ12-desaturase activity. In certain embodiments, modified commercially grown crop plants are contemplated as the transgenic organism, including, but not limited to, Arabidopsis thaliana, canola, soy, soybean, rapeseed, sunflower, cotton, cocoa, peanut, safflower, coconut, flax, oil palm, oilseed Brassica napus, corn, jojoba, Chinese tallow tree, tobacco, fruit plants, citrus plants or plants producing nuts and berries.

Methods for transforming dicotyledons, primarily by use of Agrobacterium tumefaciens and obtaining transgenic plants have been published for cotton (U.S. Pat. No. 5,004,863; U.S. Pat. No. 5,159,135; U.S. Pat. No. 5,518,908, all of which are herein incorporated by reference); soybean (U.S. Pat. No. 5,569,834; U.S. Pat. No. 5,416,011, all of which are herein incorporated by reference; McCabe, et al., 1988; Christou et al., 1988). Brassica (U.S. Pat. No. 5,463,174, herein incorporated by reference); peanut (Cheng et al., 1996, McKently et al., 1995); papaya; and pea (Grant et al., 1995).

Transformation of monocotyledons using electroporation, particle bombardment and Agrobacterium have also been reported. Transformation and plant regeneration have been achieved in asparagus (Bytebier et al., 1987); barley (Wan and Lemaux, 1994); maize (Rhodes et al., 1988; Gordon-Kamm et al., 1990; Fromm et al., 1990; Koziel et al., 1993; Armstrong et al., 1995; Toriyama et al., 1986; Part et al., 1996; Abedinia et al., 1997; Zhang and Wu, 1988; Zhang et al., 1988; Battraw and Hall, 1992; Christou et al., 1991); oat (Somers et al., 1992); orchard grass (Horn et al., 1988); rye (De la Pena et al., 1987); sugarcane (Bower and Birch, 1992); tall fescue (Wang et al., 1992) and wheat (Vasil et al., 1992; U.S. Pat. No. 5,631,152, herein incorporated by reference in its entirety).

The transformed plants are analyzed for the presence of the genes of interest and the expression level and/or profile conferred by the regulatory elements of the present invention. Those of skill in the art are aware of the numerous methods available for the analysis of transformed plants. For example, methods for plant analysis include, but are not limited to Southern blots or northern blots, PCR-based approaches, biochemical analyses, phenotypic screening methods, field evaluations, and immunodiagnostic assays. By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker proteins are known to the art and can be employed in the practice of the invention.

The seeds of the plants of this invention can be harvested from fertile transgenic plants and be used to grow progeny generations of transformed plants of this invention, including hybrid plant lines comprising the construct of this invention and expressing a gene of agronomic interest. The present invention also provides for parts of the plants of the present invention. Plant parts, without limitation, include seed, endosperm, ovule and pollen. In a particularly preferred embodiment of the present invention, the plant part is a seed. The invention also includes and provides transformed plant cells which comprise a nucleic acid molecule of the present invention.

The transgenic plant may pass along the transformed nucleic acid sequence to its progeny. The transgenic plant is preferably homozygous for the transformed nucleic acid sequence and transmits that sequence to all of its offspring upon as a result of sexual reproduction. Progeny may be grown from seeds produced by the transgenic plant. These additional plants may then be self-pollinated to generate a true breeding line of plants. The progeny from these plants are evaluated, among other things, for gene expression. The gene expression may be detected by several common methods such as western blotting, northern blotting, immunoprecipitation, and ELISA.

### Conventional Breeding

In addition to direct transformation of a particular plant genotype with a construct prepared according to the current invention, transgenic plants may be made by crossing a plant having a selected DNA of the invention to a second plant lacking the DNA. Plant breeding techniques may also be used to introduce multiple desaturases, for example Δ6, Δ12, and/or Δ15-desaturase(s) into a single plant. By creating plants homozygous for a Δ15-desaturase activity and/or other desaturase activity (e.g., Δ6- and/or Δ12-desaturase activity), beneficial metabolites can be increased in the plant.

As set forth above, a selected desaturase gene can be introduced into a particular plant variety by crossing, without the need for ever directly transforming a plant of that given variety. Therefore, the current invention not only encompasses a plant directly transformed or regenerated from cells which have been transformed in accordance with the current invention, but also the progeny of such plants. As used herein the term “progeny” denotes the offspring of any generation of a parent plant prepared in accordance with the instant invention, wherein the progeny comprises a selected DNA construct prepared in accordance with the invention. “Crossing” a plant to provide a plant line having one or more added transgenes or alleles relative to a starting plant line, as disclosed herein, is defined as the techniques that result in a particular sequence being introduced into a plant line by crossing a starting line with a donor plant line that comprises a transgene or allele of the invention. To achieve this one could, for example, perform the following steps: (a) plant seeds of the first (starting line) and second (donor plant line that comprises a desired transgene or allele) parent plants; (b) grow the seeds of the first and second parent plants into plants that bear flowers; (c) pollinate a flower from the first parent plant with pollen from the second parent plant; and (d) harvest seeds produced on the parent plant bearing the fertilized flower.

Backcrossing is herein defined as the process including the steps of: (a) crossing a plant of a first genotype containing a desired gene, DNA sequence or element to a plant of a second genotype lacking said desired gene, DNA sequence or element; (b) selecting one or more progeny plant containing the desired gene, DNA sequence or element; (c) crossing the progeny plant to a plant of the second genotype; and (d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

### Other Uses of the Present Invention

The subject invention finds many applications. One use of the sequences provided by the invention is contemplated to be the alteration of plant phenotypes, e.g., oil composition, by genetic transformation with desaturase genes. In particular embodiments, the desaturase gene is an engineered Δ15-desaturase.

For dietary supplementation, the purified PUFAs, transformed plants or plant parts, or derivatives thereof, may be incorporated into cooking oils, fats or margarines formulated so that in normal use the recipient would receive the desired amount. The PUFAs may also be incorporated into edible compositions such as infant formulas, nutritional supplements or other food products, and may find use as anti-inflammatory or cholesterol lowering agents. The purified PUFAs, transformed plants or plant parts may also be incorporated into animal, particularly livestock, feed.

Having now generally described the invention, the same will be more readily understood through reference to the following examples which are provided by way of illustration, and are not intended to be limiting of the present invention, unless specified. Each periodical, patent, and other document or reference cited herein is herein incorporated by reference in its entirety.

The following examples are included to demonstrate preferred embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention.

### EXAMPLES

The following examples are included to illustrate embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Bioinformatics Analysis and Molecular Engineering of Delta-15 Desaturase Sequences

Sequence alignments of delta-15 desaturases from various sources, including Mortierella alpina, Neurospora crassa, Saccharomyces kluyveri, Aspergillis nidulans and Chlorella vulgaris, reveal regions of the molecules that comprise highly variable amino acid sequences in addition to more conserved regions. The regions of high diversity were selected for molecular engineering experiments for the purpose of generating molecules with novel characteristics, such as substrate preference and/or enzymatic activity. Using the Mortierella alpina delta-15 desaturase protein as a parent protein, changes were designed in these highly variable regions to sample from the diversity observed in naturally occurring delta-15 desaturases. Additional conservative amino acid substitutions were included in the designs as well.

Polynucleotide sequences were engineered to correspond to the amino acid variants designed from the bioinformatics analysis. For some variants, generation of the polynucleotide sequences was executed using a novel Degenerate Oligonucleotide Tail (DOT) approach, disclosed in U.S. patent application Ser. No. 11/827,318, herein incorporated by reference in its entirety. Briefly, a pair of oligonucleotides were designed that to anneal to the plasmid template on the other side and adjacent to the targeted region, and were capable of serving as primers in a polymerase chain reaction. The oligonucleotides comprised a modification, such as 2′-O-methylribose, that is capable of terminating extension by a polymerase, thereby leaving the PCR product with single-stranded tails. The tails, located 5′ of the terminating base, were designed such that they may anneal to each other. To introduce variation into the targeted region, the tails included degenerate base positions. The primers that introduced the desired diversity into the engineered delta-15 desaturase were designed and ordered from Operon Technologies, Inc. (Alameda, Calif.). Sets of DOT primers were used to introduce mutations into the delta-15 desaturase molecule by means of terminated PCR on the template (yeast-codon optimized sequence in a pYES2.1 vector).

For some cases, more than one iteration of the DOT method was employed to generate the engineered delta-15 desaturase molecular variants. Upon creation of variants in a single DOT region, the resultant molecules were used as templates for generation of variants in a second DOT region. Another option would be to select a set of molecules from one DOT region, combine them, or use them individually as templates for another DOT variation region. It is contemplated that many different combinations and iterations of the DOT method may be utilized to generate any number of molecular variant types.

Other variants were created as chimeric molecules via gene splicing. Other methods known in the art could be used to engineer the molecules of the present invention.

PCR was then performed according to methods well known in the art, with Pfu and Pfu Turbo polymerase mixtures using the following thermocycler gradient program:

TABLE 1
PCR Thermocycler Parameters for DOT method generation
of delta-15 desaturase engineered variants

| | |
|---|---|
| Temperature (° C.) | Time |

One Cycle

| | | |
|---|---|---|
| 95 | 5 | minutes |
| 50-65 | 3 | minutes |
| 72 | 12 | minutes |

30 Cycles

| | | |
|---|---|---|
| 95 | 45 | seconds |
| 50-65 | 45 | seconds |
| 72 | 12 | minutes |

Final Step

| | |
|---|---|
| 4 | hold |

Resulting PCR products were treated with DpnI to remove the parental template molecules, then were self-annealed, transformed into chemically competent E. coli Top10 (Invitrogen) and plated onto solid CircleGrow medium with ampicillin. The individual colonies were grown in liquid culture and the DNA was isolated by a standard miniprep procedure using methods well known in the art. The plasmid DNA was sequenced using the BigDye DNA sequencing kit and two primers (Gal1 and V5) to cover the whole sequence of the desaturase gene.

### Example 2

### Yeast Cell Transformation and Expression

The pYES2.1/V5-His clones comprising the engineered delta-15 desaturases were introduced into a host strain Saccharomyces cerevisiae INVSc1 (auxotrophic for uracil) (Invitrogen) using the PEG/Li Ac protocol as described in the Zymos EZ yeast transformation manual. Transformants were selected on plates made of SC minimal media minus uracil with 2% glucose. Colonies of transformants were used to inoculate 800 μl of SC minimal media minus uracil and 2% raffinose grown overnight at 30° C. For induction, stationary phase yeast cells were diluted in SC minimal media minus uracil supplemented with 2% galactose and 2% raffinose grown for 2 days at 15° C. or 16 hours at 30° C. When exogenous fatty acids were provided to the cultures, 0.005% (v/v) LA (Δ9,12-18:2) and 0.005% (v/v) GLA (Δ6,9,12-18:3) were added with the emulsifier 0.1% Tergitol. The cultures were grown for 2 days at 15° C. or 16 hours at 30° C., and subsequently harvested by centrifugation. Cell pellets were washed once with sterile water, to remove the media, and lyophilized to dryness. The host strain transformed with the vector containing the LacZ gene was used as a negative control in all experiments.

### Example 3

### Functional Yeast Cell Based Fatty Acid Desaturase Assay

To characterize the substrate selectivity and relative activity of fatty acid desaturase enzymes, a yeast (Saccharomyces cerevisiae) cell-based assay system was developed. Yeast is a suitable host system for studying desaturase enzymes as it is incapable of endogenously producing polyunsaturated fatty acids, as it only naturally expresses a delta-9 desaturase. The fatty acid compositional profile of yeast harboring an introduced desaturase gene was obtained through fatty acid methyl ester gas chromatographic separation coupled to flame ionization detection.

Lipids were extracted from lyophilized yeast pellets and converted to fatty acid methyl esters (FAMEs) by adding 0.05 mL toluene containing an internal standard and 0.167 mL of 5% (v/v) sulfuric acid in methanol and heating to 90° C. for 90 minutes. The FAMEs were extracted by addition of 0.3 mL 10% (w/v) NaCl and 0.3 mL of heptane. The autosampler needle penetration depth was set to sample from the heptane layer containing the FAMEs and used directly for gas chromatography (GC). The FAMEs were identified on a Hewlett-Packard 6890 II Plus GC (Hewlett-Packard, Palo Alto, Calif.) equipped with a flame-ionization detector and a capillary column (omegawax 250; 15 m×0.25 mm i.d.×0.25 μm; Supelco, Bellefonte, Pa.). A 30:1 split ratio was used for injections. The injector was maintained at 250° C. and the flame ionization detector was maintained at 270° C. The column temperature was maintained at 190° C. for 0.1 min following injection, increased to 240° C. at 50° C./min, and held at 240° C. for 0.75 min.

The results shown in Table 2 demonstrate that the native M. alpina delta-15 desaturase exhibits Δ15 desaturase activity in a yeast expression system. The substrate preference was deduced from a yeast induction assay, whereby yeast cultures induced to express recombinant desaturase are fed equal amounts of LA and GLA. Substrate preference for GLA over LA is calculated by measuring the amounts of their respective products, SDA and ALA, and using the following formula:

Preference Ratio=(SDA/(SDA+GLA))/(ALA/(LA+ALA),

where SDA is stearodonic acid,

GLA is gamma linolenic acid,

ALA is alpha linolenic acid,

and LA is linoleic acid.

The yeast incorporated these fatty acids into their membranes where they became substrates for the recombinant desaturase. The products of LA and GLA Δ15 desaturation are ALA and SDA, respectively. Four individual MaD15D colonies were selected and provided LA and GLA, with a substrate selectivity for GLA that is 1.2 fold higher than for LA. The negative control was a pYES2.1 vector comprising a LacZ insert.

TABLE 2
Delta 15 desaturase activity of M. alpina in a yeast expression system.

| | FA In | | | | | Substrate |
|---|---|---|---|---|---|---|
| Construct | Medium | LA | GLA | ALA | SDA | Preference* |
| neg control | — | 0 | 0 | 0 | 0 | NA |
| neg control | — | 0 | 0 | 0 | 0 | NA |
| neg control | LA + GLA | 7.53 | 10.84 | 0 | 0 | NA |
| neg control | LA + GLA | 8.97 | 8.70 | 0 | 0 | NA |
| neg control | LA + GLA | 8.04 | 7.67 | 0 | 0 | NA |
| neg control | LA + GLA | 6.97 | 7.34 | 0 | 0 | NA |
| MaD15D | — | 0 | 0 | 0 | 0 | NA |
| MaD15D | — | 0 | 0 | 0 | 0 | NA |
| MaD15D | LA + GLA | 5.94 | 5.21 | 4.50 | 6.21 | 1.26 |
| MaD15D | LA + GLA | 5.46 | 4.17 | 4.78 | 6.08 | 1.27 |
| MaD15D | LA + GLA | 5.88 | 3.96 | 5.16 | 5.81 | 1.27 |
| MaD15D | LA + GLA | 5.27 | 3.93 | 4.82 | 5.87 | 1.26 |

Engineered desaturases of the present invention that were designed and expressed according to the methods described above were tested in a similar manner in this cell-based yeast expression system. SEQ ID NO: 1 through SEQ ID NO: 331 are engineered delta-15 desaturases that exhibited assay activity greater than that seen from the native M. alpina delta-15 desaturase in the cell-based yeast expression system. SEQ ID NO: 332 through SEQ ID NO: 662 are the corresponding delta-15 desaturase nucleotide molecules that encoded the engineered proteins that exhibited assay activity greater than that seen from the native M. alpina delta-15 desaturase in the cell-based yeast expression system. SEQ ID NO: 663 through SEQ ID NO: 722 are engineered delta-15 desaturases that exhibited assay comparable to that of the native M. alpina delta-15 desaturase in the cell-based yeast expression system. SEQ ID NO: 783 through SEQ ID NO: 842 are the corresponding delta-15 desaturase nucleotide molecules that encode the engineered proteins that exhibited assay comparable to that of the native M. alpina delta-15 desaturase in the cell-based yeast expression system. SEQ ID NO: 843 through SEQ ID NO: 902 are the corresponding delta-15 desaturase nucleotide molecules that encode the engineered proteins that were not active in the cell-based yeast expression system.

### Example 4

### Soybean Somatic Embryo Transformation

Evaluation of oil composition in transgenically altered soy may be achieved using a soy somatic embryogenesis system. This system uses the ability to generate somatic embryos through the use of embryogenic cell cultures derived from the cotyledons of immature soy embryos. As practiced, transformation of said embryos occurs by introduction of the effector gene or genes through particle bombardment. Transformed embryos are selected by introducing a gene for NptII on the plasmid containing the effector gene(s) and by using paromomycin in the growth medium. Transgenic embryos are matured on a maturation medium, grown for a period of time, harvested, frozen in liquid nitrogen and analyzed for oil composition using methods known in the art.

### Example 5

### Transformation of Plants with an Engineered Delta-15 Desaturase Gene

This example describes the transformation and regeneration of transgenic Arabidopsis thaliana plants expressing a heterologous Δ15-desaturase coding sequence. Transformation vectors comprising an engineered delta-15 desaturase coding sequence are introduced into Agrobacterium tumefaciens strain ABI using methodology well known in the art. Transgenic A. thaliana plants are obtained as described by Bent et al. (1994) or Bechtold et al. (1993). Briefly, cultures of Agrobacterium with the vectors comprising the engineered desaturase coding sequences, along with a selectable marker such as CP4, are grown overnight in LB (10% bacto-tryptone, 5% yeast extract, and 10% NaCl with kanamycin (75 mg/L), chloramphenicol (25 mg/L), and spectinomycin (100 mg/L)). The bacterial culture is centrifuged and resuspended in 5% sucrose+0.05% Silwet-77. The aerial portion of whole A. thaliana plants (−5-7 weeks of age) are immersed in the resulting solution for 2-3 seconds. The excess solution is removed by blotting the plants on paper towels. The dipped plants are placed on their side in a covered flat and transferred to a growth chamber at 19° C. After 16 to 24 hours the dome is removed and the plants are set upright. When plants reached maturity, water is withheld for 2-7 days prior to seed harvest. Harvested seed is passed through a stainless steel mesh screen. To select transformants, seed is plated on agar medium containing 50 mg/L glyphosate. Green seedlings are rescued and transplanted into 4″ pots and grown under the conditions described above. Leaves were harvested for fatty acid analysis when the rosette was at the 4-leaf stage. After lyophilization, leaf fatty acids were analyzed as described above.

In order to assess the functional specificity of a delta-15 desaturase clone to direct production of ALA in seeds, the coding region is cloned into a seed-specific expression vector in which a seed-specific promoter drives expression of the transgene. The resulting construct is transformed into A. thaliana and seeds of transformed T2 plants are analyzed for fatty acid composition.

### Example 6

### Activity of an Engineered Delta 15-Desaturase in Combination with Delta 6- and Delta 12-Desaturases

The activity of the engineered Δ15-desaturase, in combination other desaturase genes, such as Δ6- and Δ12-desaturases, from either a native or engineered source, may be evaluated by transforming a plant with a construct comprising the engineered delta-15 desaturase coding sequence with additional desaturase genes, for example a delta-6 desaturase and/or a delta-12 desaturase, under the control of a seed-specific promoter, such as the napin promoter. Fatty acid content of 10-seed pools from individual R0 plants may be determined using methods known in the art. The levels of stearic acid (18:0) (SA), oleic acid (18:1) (OA), LA, ALA, SDA and GLA are then evaluated.

### Example 7

### EPA Equivalence

One measure of seed oil quality for health value is EPA equivalence (James et al., Metabolism of stearidonic acid in human subjects: comparison with the metabolism of other n-3 fatty acids, Am J Clin Nutr 77:1140-5, 2003 and U.S. Pat. No. 7,163,960, herein incorporated by reference in its entirety). The value reflects the metabolic conversion rate to EPA. This is calculated by adding the % ALA divided by 14 and the % SDA divided by 4. The oil compositions obtained from seeds expressing the desaturases of the present invention may be determined and the EPA equivalence calculated.

An example of the analysis is given by comparison of conventional canola oil relative to an example of a typical high SDA oil composition of 10% ALA and 15% SDA. Canola oil from conventional varieties has approximately 12% ALA and 0% SDA and thus has an EPA equivalence of 12/14+0/4=0.8. In contrast, the high SDA oil composition example has an EPA equivalence of 10/14+15/4=4.4. Values are by wt %, not on a serving basis. The vast difference shows the importance of producing SDA in canola oil.

### REFERENCES

Abedinia et al., Aust. J. Plant Physiol. 24:133-141 (1997)
Armstrong et al., Crop Science 35:550-557 (1995)
Arondel et al., Science 258(5086):1353-1355 (1992)
Ausubel et al., Current protocols in molecular biology. John Wiley and Sons Inc., New York, N.Y. (1987)
Baggio et al., Nephron 91:3 (2002)
Bairati et al., Circulation 85:950-956 (1992)
Battraw and Hall, Plant Sci. 86:191-202 (1992)
Bower and Birch, Plant J. 2:409 (1992)
Bray et al., Planta 172(3): 364-370 (2004)
Bustos et al., EMBO J. 10: 1469-1479 (1991)
Bytebier et al., Proc. Natl. Acad. Sci. (USA) 84:5354 (1987)
Cheng et al., Plant Cell Rep. 15:653-657 (1996)
Chou and Fasman, Adv Enzymol Relat Areas Mol Biol 47:45-148 (1978)
Christou et al., BioTechnology 9:957 (1991)
Christou et al., Plant Physiol. 87:671-674 (1988)
Cleland and James, J Rheumatol 27(10):2305-7 (2000)
De Deckerer, Eur. J. Clin. Nutr., 52:749 (1998)
De la Pena et al., Nature 325:274 (1987)
Diaz et al., J. Biol. Chem., Vol. 277, Issue 50, 48099-48106 (2002)
Doyle et al., J Biol Chem 261: 9228-9238 (1986)
Fromm et al., BioTechnology 8:833 (1990)
Goeddel, Methods Enzymol. 185:3-7 (1990)
Gordon-Kamm et al., Plant Cell 2:603-618 (1990)
Grant et al., Plant Cell Rep. 15:254-258 (1995)
Hamilton et al., Proc Natl Acad Sci USA 93(18):9975-9979 (1996)
Haymes et al., Nucleic Acid Hybridization, A Practical Approach, IRL Press, Washington, D.C. (1985)
Horn et al., Plant Cell Rep. 7:469 (1988)
James et al., Am J Clin Nutr 77:1140-5 (2003)
Knipple et al., Genetics 162:1737-1752 (2002)
Knutzon et al., Proc. Natl. Acad. Sci U.S.A. 89: 2624-2628 (1992)
Koziel et al., BioTechnology 11:194 (1993)
Kridl et al., Seed Sci. Res. 1: 209 (1991)
Kyte and Doolittle. J Mol Biol 157(1):105-132 (1982)
Lam and Chua, Science 248: 471 (1991)
Lindstrom et al., Developmental Genetics 11: 160 (1990)
Mantzioris et al., Am J Clin Nutr. 59(6): 1304-9 (1994)
McCabe et al., Biotechnology, 6: 923 (1988)
McKently et al., Plant Cell Rep. 14:699-703 (1995)
Meesapyodsuk et al., Biochemistry, 39(39), 11948-11954 (2000)
Nagakura et al., Eur Respir J 16: 861-865 (2000)
Naylor et al., Nature 405, 1017-1024 (2000)
Needleman and Wunsch, J Mol Biol 48(3):443-53 (1970)
Part et al., Plant Mol. Biol. 32:1135-1148 (1996)
Pearson and Lipman, Science 227(4693):1435-1441 (1985)
Pesole et al., Biotechniques 25(1):112-7, 120-3 (1998)
Reed et al., Plant Physiol 122(3): 715-720 (2000)
Rhodes et al., Science 240:204 (1988)
Sambrook and Russel, Molecular Cloning, A Laboratory Manual, Editions 1-7, Cold Spring Harbor Press, Cold Spring Harbor, N.Y. (1989-2007)
Sasata et al., J Biol Chem 279(38): 39296-39302 (2004)
Simopoulos, Can J Physiol Pharmacol. 75(3):234-9 (1997)
Smith and Waterman, J Mol Biol 147(1):195-7 (1981)
Somers et al., BioTechnology 10:1589 (1992)
Søyland et al., Eur J Clin Invest. 24(4):236-42 (1994)
Sperling et al., Prostaglandins, Leukotrienes and Essential Fatty Acids 68(2):73-9 (2003)
Tierney et al., Planta 172:356 (1987)
Toriyama et al., Theor Appl. Genet. 205:34 (1986) Vasil et al., BioTechnology 10:667 (1992)
Vodkin et al., Cell, 34: 1023 (1983)
Wan and Lemaux, Plant Physiol 104:37 (1994)
Wang et al., BioTechnology 10:691 (1992)
Wolk, et al., Proc Natl Acad Sci USA 81(5): 1561-1565 (1984)
Zhang and Wu, Theor. Appl. Genet. 76:835 (1988)
Zhang et al., Plant Cell Rep. 7:379 (1988)

We claim:

1. An engineered fatty acid desaturase molecule, wherein said desaturase molecule:
a. exhibits a substrate preference for Gamma Linolenic Acid (GLA) over Linoleic Acid (LA) of at least 1.75× and as calculated by the formula (SDA/(SDA+GLA))/(ALA/(LA+ALA), where SDA is stearodonic acid, GLA is gamma linolenic acid, ALA is alpha linolenic acid, and LA is linoleic acid; or
b. exhibits a total conversion rate of GLA to SDA of at least 40%; or
c. when expressed in a transgenic plant, causes the transgenic plant to produce more omega-3 fatty acid than non-transgenic plants; or
d. when co-expressed with a delta-6 fatty acid desaturase in a transgenic plant, causes the transgenic plant to accumulate, as compared to a non-transgenic plant, a condition selected from the group consisting of: more SDA than ALA, and greater conversion of GLA to SDA than LA to ALA.

2. The desaturase molecule of claim 1, further defined as a molecule that desaturates a fatty acid molecule at carbon 15.

3. The desaturase molecule of claim 1, wherein said molecule has 80% similarity to a fungal desaturase.

4. The desaturase molecule of claim 1, wherein said molecule comprises amino acid sequence variants generated from a parental fungal desaturase.

5. The desaturase molecule of claim 1, wherein said desaturase is identified from a genus selected from the group consisting of: Mortierella, Neurospora, Aspergillus, Saccharomyces, Botrytis, Chlorella.

6. The desaturase molecule of claim 1, wherein the molecule has a sequence selected from the group consisting of SEQ ID NO: 1 through SEQ ID NO: 331.

7. The desaturase molecule of claim 1, wherein the molecule exhibits a percent sequence identity of greater than about 90% identity with a molecule selected from the group consisting of: SEQ ID NO: 1 through SEQ ID NO: 331.

8. The desaturase molecule of claim 1, wherein the molecule comprises a fragment of SEQ ID NO: 1 through SEQ ID NO: 331.

9. A polynucleotide encoding the desaturase molecule of claim 1.

10. The polynucleotide of claim 9, wherein the polynucleotide has a sequence selected from the group consisting of SEQ ID NO: 332 through SEQ ID NO: 662.

11. The polynucleotide of claim 9 that, when under the control of a regulatory element, is capable of expression in a plant.

12. The polynucleotide of claim 9, or any complement thereof, or any fragment thereof, comprising a nucleic acid sequence that exhibits a substantial percent sequence identity of greater than about 90% to a sequence selected from the group consisting of SEQ ID NO: 332 through SEQ ID NO: 662.

13. A construct comprising the polynucleotide of claim 9.

14. The construct of claim 13, further comprising a second polynucleotide that is transcribable.

15. The construct of claim 14, wherein the second transcribable polynucleotide molecule is selected from the group consisting of: a non-coding regulatory element, a selectable marker, a gene encoding a second desaturase, and a gene of agronomic interest.

16. The construct of claim 15, wherein the gene of agronomic interest is a gene controlling the phenotype of a trait selected from the group consisting of: herbicide tolerance, insect control, modified yield, fungal disease resistance, virus resistance, nematode resistance, bacterial disease resistance, plant growth and development, starch production, modified oils production, high oil production, modified fatty acid content, high protein production, fruit ripening, enhanced animal and human nutrition, biopolymers, environmental stress resistance, pharmaceutical peptides and secretable peptides, improved processing traits, improved digestibility, enzyme production, flavor, nitrogen fixation, hybrid seed production, fiber production, and biofuel production.

17. A host cell stably transformed with the construct of claim 15.

18. The host cell of claim 17, further defined as a plant cell.

19. A progeny of the host cell of claim 18, wherein said progeny has inherited the polynucleotide of said polynucleotide construct.

20. The plant cell of claim 18, wherein said plant cell is a cell of a plant selected from the group consisting of: Arabidopsis thaliana, Brassica napus, Brassica rapa, rapeseed, sunflower, safflower, canola, corn, soybean, cotton, flax, jojoba, Chinese tallow tree, tobacco, cocoa, peanut, fruit plants, citrus plants, plants producing nuts, plants producing seeds, and plants producing berries.
